(19) United States
(12) Patent Application Publication
(10) Pub. No.: US 2012/0237566 A1
(43) Pub. Date: Sep. 20, 2012

(54) INHIBITING STOMACH-ACID RELEASE, REDUCING INFLAMMATION AND PREVENTING AND TREATING CANCER: COMPOSITIONS AND METHODS OF USE

(75) Inventors: Tushar K. Ray, Tempe, AZ (US); Clyde G. Hanson, Minneapolis, MN (US); Paul Burgio, St. Paul, MN (US)

(73) Assignee: Venture Isles, LLC, Minneapolis, MN (US)

(21) Appl. No.: 13/421,650

(22) Filed: Mar. 15, 2012

Related U.S. Application Data
(60) Provisional application No. 61/453,092, filed on Mar. 15, 2011.

(51) Int. Cl. A61K 31/26; A61P 1/00; A61P 29/00; A61K 33/24; A61K 9/00; A61K 33/00; A61K 33/10; A61K 33/08; A61K 31/4439; A61P 35/00

(52) U.S. Cl. 424/400; 514/514; 514/338; 424/715; 424/687; 424/691; 424/692; 424/653; 424/717

(57) ABSTRACT

The present invention relates to improved methods of preventing or inhibiting the release of acid in the stomach, which are uniquely reversible at the cellular level, through the use of organic isothiocyanates and thiocyanates with divalent cations such as barium, zinc and calcium for human and veterinary applications. The present invention also relates to methods of preventing or treating persistent chronic gastritis, GERDs, ulcer and or stomach cancer by inhibiting the release of stomach acid and by reducing inflammation with fewer and milder side-effects expected than current treatments. A method of screening therapeutics is described.

### CROSS-REFERENCE TO RELATED APPLICATIONS

The present invention claims priority to U.S. Provisional application Ser. No. 61/453,092, filed Mar. 15, 2011, incorporated by reference herein.

### BACKGROUND

The present invention generally relates to methods of preventing or inhibiting the release of acid in the stomach through the use of isothiocyanates and thiocyanates in combination with divalent ions as barium, zinc and calcium. The present invention also generally relates to methods of preventing or treating persistent chronic gastritis and ulcers by inhibiting the release of stomach acid and also preventing or treating cancer throughout the body by causing anti-inflammatory activity that reduces mitosis. The field of the invention includes the option of delivering the formulation in a system that provides prolonged delivery over time in the stomach for optimal clinical benefit.

Acid is normally released into the stomach to aid in digestion; however, the release of too much acid or acid released into the empty stomach can cause chronic irritation of the stomach lining, ulcers, and acid reflux disease. Many oral treatments are strong bases and are intended to neutralize the acid after it has been released. This type of treatment can still lead to chronic irritation and its consequences over repeated occurrences. There is a clinical need for a pharmaceutical or food supplement to prevent the release of the acid and to eliminate the associated negative consequences and also to provide a reversible inhibition of acid secretion so the subject's acid levels can be optimized much faster for normal digestion and nutrition.

Heartburn, also known as GERD (gastro-esophageal reflux disease), occurs when the valve in the esophagus (the LES or lower esophageal sphincter) becomes weak and instead of acting as a one-way valve, it lets stomach contents flow back into the esophagus. Usually, after glutition, the LES opens to let food into the stomach before closing to prevent acid from returning into the esophagus. The backward flow of acid coming from this acid reflux is often noticed as a burning sensation that is called heartburn. Therefore, acid reflux patients often experience a particular burning feeling under the breastbone, often coming at night.

Gastro-esophageal reflux disease is a menacing condition necessitating immediate diagnosis and treatment. If unattended, GERD can badly corrode the lining of the esophagus causing continual inflammation and possibly even esophageal cancer. Persistent with its concomitant symptoms of heartburn can be distracting and painful enough to ruin a person's daily activities and seriously impinge on the esophagus. The high prevalence of GERD and other conditions caused by excess acid clearly demonstrate that there is an unmet need for a medication that prevents the release of too much stomach acid.

Direct and permanent inhibition of the proton pump (gastric H, K-ATPase) by ingestion of the pump inhibitors is currently a popular way to treat stomach hypersecretion. The leading over-the-counter proton-pump inhibitor (PPI) on the market today is Prilosec™. This drug turns off the proton pumps (see below) of the acid secreting cell for the life of the cell. Stomach acid is needed for normal food digestion and absorption. There is a lengthy list of side effects of PPI that include diarrhea, gas, headache, nausea, stomach pain, taste perversion, various allergic reactions, chest pain, seizure and the list goes on (see www.drugs.com). So, there is a dire need of a drug with fewer and milder side-effects to handle the GERD-related problems worldwide.

Clinically used proton-pump inhibitors (PPI) include the following: Omeprazole (brand names: Losec, Prilosec, Zegerid, ocid, Lomac, Omepral, Omez); Lansoprazole (brand names: Prevacid, Zoton, Inhibitol, Levant, Lupizole); Dexlansoprazole (brand name: Kapidex, Dexilant); Esomeprazole (brand names: Nexium, Esotrex); Pantoprazole (brand names: Protonix, Somac, Pantoloc, Pantozol, Zurcal, Pan); Rabeprazole (brand names: Zechin, Rabecid, Aciphex, Pariet, Rabeloc). All PPI molecules named above share the same Benzimidazole nucleus as given for Omiperazole, as shown in FIG. 1.

Under acidic environment of the gastric lumen the drug molecule undergoes spontaneous intramolecular rearrangement making them highly reactive to thiol (—SH) groups in close contact. The secretary-membrane that has a gastric H, K-ATPase pump is known to rely on a critical cysteine thiol (—SH) for function. At low pH, the omiperazole derivatives covalently binds to the critical thiol moiety in question thus inactivating the enzyme, as shown in FIG. 2.

Like the Prilosec™ (the chemical structure is shown above), all PPI have the same basic Benzimidazole nucleus linked to a S-atom and the drugs attack the critical —SH group of the H, K-ATPase permanently inhibiting the proton pump, as shown in FIG. 2. There are other proton-transporting mechanisms (provided below) within a cell that also depend on critical thiol (—SH) for activity and function, and hence are likely to be affected by the PPI molecules in contact with their respective acidic microenvironment causing various side-effects. The mechanism of PPI action is shown in FIG. 2.

The Demerits of the PPI treatment include the following: (1) Since the parietal or acid secreting cells of the stomach are fully differentiated the PPI molecule conjugated covalently (shown in FIG. 2 below) to the proton pump stays there permanently until replaced by a new parietal cell with fresh pump sites, which would most likely have adverse side effects in long-term users. (2) Also, similar to the gastric proton pump the non-gastric H, K-ATPase system of the distal colon and kidney tubules (Sangan et al, 1997) will be likewise be chronically affected by these types of drugs. (3) Another proton transporting system, the ubiquitous Na/H exchanger, well-known to be dynamic in intracellular pH regulation also depends on a critical thiol group (Grinstein, 1985) for proper function. Hence, long-term treatment with the PPI molecule would likely affect the Na/H exchanger system of the cells including the neurons (Luo et al, 2005) and the heart cells (Crinoline and Ennis, 2007). (4) Still another type of proton pump (called V-type H-ATPase) that depends on a critical SH-group (D'Souza et al, 1987) for function is located on the lysosomes within a cell. The pump helps to maintain intra-lysosomal acidity (pH 5.0) for recycling the cellular garbage-material following acid digestion. Naturally, inhibition of the V-type proton pump will be harmful for any cell. The PPI molecule entering into a cell is likely to interfere with the lysosomal activity causing bad side-effects. (5) The preceding information given in 1, 2, 3 and 4 (above) could help us explain many of the diverse side-effects of the PPI treatment mentioned earlier and reported in www.drugs.com. (6) Furthermore, the low pH of the stomach normally offers a strong line of defense by killing harmful bacteria that enters into our body with the ingested foodstuffs. Regular use of PPI adversely affects this defense mechanism.

In contrast to PPI, the use of isothiocyanates and thiocyanates in combination with divalent ions such as barium, zinc and calcium only temporarily stop the proton pumps of the parietal cell but still limit stomach acid secretion. The acid secretion and the proton pump resume normal function when the active ingredients are removed from contact with the cell. The length of time in contact with the cell can be specified by the design of the medication delivery system. Additional benefits are the anti-inflammatory and anti-mitotic effects and thus the invention also provides an anti-cancer benefit.

### SUMMARY OF THE INVENTION

The anti-inflammatory formulation comprises a combination of a plurality of actives contained in one unit of a delivery system custom designed to reach the target tissue. The combination of the plurality actives can be simultaneously released at the extracellular surface of the cell membrane or in the cell to act in concert to achieve an improved clinical result. The combination of the plurality of actives include at least one or all of the following: green tea, curcumin, thiocyanate (TC), isothiocyanate (ITC) and zinc. Green tea, curcumin, garden cress, papaya, thiocyanate (TC), isothiocyanate (ITC) and zinc includes an anti-inflammatory effect and each achieves this result through a different physiochemical pathway throughout the body. Zinc has a potentiating effect on TC and ITC to reduce inflammation; therefore the plurality of actives acting on a single cell simultaneously has a heightened effect over a single agent to reduce inflammation. Reduced cellular inflammation also reduces the mitotic activity of the cell and supports an anti-cancer benefit.

The present invention relates to a method of treating a subject having excess secretion of stomach acid comprising the administration of isothiocyanates or thiocyanates in a formulation to enhance the inhibition of the release of acid. In one embodiment, the formulation includes a component such as calcium carbonate that can neutralize already secreted acid. In another embodiment, isothiocyanates or thiocyanates act synergistically with a plurality of divalent ions to provide an enhanced clinical effect.

The present invention also relates to a method of combining one or more ATPase inhibiting compounds, including, but not limited to, divalent ions with isothiocyanates or thiocyanates in a combination of zinc with calcium or barium to provide an enhanced clinical benefit to the subject.

The present invention also relates to a method of treating unwanted inflammation throughout the body.

The present invention also relates to a method of treating a subject with cancer comprising the administration of a thiocyanate formulation to the subject to stop the rapid mitosis of a tumor.

The present invention also relates to a method of treating a subject with clinical conditions of the small and large intestine, where down regulating the Na, K-ATPase system includes a therapeutic effect.

The present invention also relates to rational anti-ulcer and antisecretary drug design based on their binding affinity to highly purified gastric H, K-ATPase preparation in the presence and absence of K ions, and the K-sensitive ligands are classified as either inhibitor or uncoupler of the pump.

The present invention also relates to testing the anti-cancer activity of the aforementioned anti-ulcer agents using cancer cells in tissue culture, when a clear sign of anti-mitotic activity upon long-term (days) exposure to ligand broadens its therapeutic benefits.

The methods, systems, and compositions are set forth in part in the description which follows, and in part will be obvious from the description, or can be learned by practice of the methods, compositions, and systems. The advantages of the methods, compositions, and systems will be realized and attained by means of the elements and combinations particularly pointed out in the appended claims. It is to be understood that both the foregoing general description and the following detailed description are exemplary and explanatory only and are not restrictive of the methods, compositions, and, systems, as claimed.

### BRIEF DESCRIPTION OF THE DRAWINGS

In the accompanying figures, like elements are identified by like reference numerals among the several preferred embodiments of the present invention.

FIG. 1 is a chemical structure of Omiperazole (or Prilosec™)

FIG. 2 is a chemical reaction of the spontaneous intra-molecular rearrangement of the Omiperazole under acidic environment of the stomach producing an intermediate species that is highly reactive to thiol (—SH), and resultant inactivation of the secretary membrane located H, K-.

ATPase pump in the immediate vicinity

FIG. 3: is a Dual-Topology Model for the univalent cation transporting ATPase system showing bilayer orientation of the a and 13 subunits.

FIG. 4 is a flow chart of one embodiment of the Drug Screening Process.

FIG. 5a is a schematic diagram of the catabolism of Glucosinolates by native Myrosinase following damage or disintegration of the cells in cruciferous vegetables generating various isothiocyanates; and FIG. 5b is chemical structures of various isothiocyanates.

FIG. 6A is a table for a method of treating inflammation or cancer with oral delivery of food supplement or drug through a layered tablet; and FIG. 6B is a schematic diagram of one of two or three layers of compressed powder in the 3-Layer Tablet.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

The present invention relates to methods of preventing or inhibiting the release of acid in the stomach through the use of organic isothiocyanates and thiocyanates with divalent cations such as barium, zinc and calcium for human and veterinary applications, which are uniquely reversible at the cellular level. The formulation can also include green tea and/or curcumin to provide an enhanced clinical benefit since all contribute an anti-inflammatory effect and each achieves this result through a different physiochemical pathway. In alternative embodiments, methods of preventing or treating persistent chronic gastritis, GERDs, ulcer and or stomach cancer are disclosed herein by inhibiting the release of stomach acid and by reducing inflammation with fewer and milder side-effects expected than current treatments, such as proton-pump inhibitors like Prilosec OTC® and Prevacid®. The formulation may be delivered through food supplements and additives; over-the-counter medicines; and prescription drugs. A method of screening therapeutics is also described herein.

Generally speaking, the invention comprises compounds limiting the ATPase function (or acting as an uncoupler) including the compound with the Formula (I):

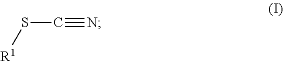

Alternatively, the invention comprises compounds limiting the ATPase function (or acting as an uncoupler) including the Formula (II):

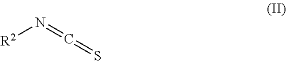

wherein R1 or R2 is any organic compound containing carbon, including, but not limited the following: independently hydrogen, alkyl, saturated alkyl, unsaturated alkyl, aryl, halo, nitro, amino hydroxyl, alkoxy, alkylamino, dialkylamino, alkylthio, alkyloxy, alkylhydrazino, alkylcarbonyl, alkylcarboxy, alkylcarbonylamino, alkylcarbonylhydrazino, cycloalkylcarbonyl, a straight-chain or branched saturated or unsaturated alkyl or an aryl, arylalkoxy carbonyl, arylalkyl, arylcarbonyl, arylalkylcarbonyl, aryloxy, aryloxycarbonyl, arylthio, arylamino, arylhydrazino, arylcarboxy, arylcarbonylamino, arylcarbonylhydrazino, arylalkanoyl, aroyl, aryloxyalkanoyl, heterocyclyl, heteroaryl, heterocycle, heterocyclic, saturated heterocyclic, heterocyclyloxycarbonyl, heterocyclylalkanoyl, heterocyclylalkoxycarbonyl, an oxime, or an oxime methyl, an unsubstituted or substituted aryl substituted selected from the group consisting of a halogen, a nitro, an amino, a hydroxyl, a thio, an acyl, an alkyl, and a cyano; an aromatic ring of formula (II) is unsubstituted or substituted with at least one substituent selected from the group consisting of a halogen, a nitro, an amino acid, a hydroxyl, a thio, an acyl, an alkyl, and a cyano; pharmaceutically acceptable salts, esters, and prodrugs thereof, carbohydrates and their derivatives, nucleic acids and their derivatives, steroids, fats, fatty acids, or oils, antigens, antibodies, enzymes, hormones, neurotransmitters, proteins and peptides, lectins, vitamins, and preferably R1 and R2 is an organic molecule. Preferably, R1 or R2 is an organic and lipid soluble molecule. Lipid soluble refers to the ability of a molecule to dissolve in fats, oils, lipids, and non-polar solvents, such as lipophilicity or hydrophobic properties that interact within themselves and with other substances through the London dispersion forces.

In certain embodiments, Formula (I) and (II) or a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, a mixture of diastereomers, or a pharmaceutically acceptable salt, solvate, or prodrug thereof.

The present invention is intended to include all isotopes of all atoms occurring in the present compounds. Isotopes include those atoms having the same atomic number but different mass numbers. By way of general example and without limitation, isotopes of hydrogen include (D) and tritium (T). Isotopes of carbon include ¹³C and ¹⁴C. Isotopes of sulfur include ³²S, ³³S, ³⁴S, and ³⁶S. Isotopes of nitrogen include ¹⁴N and ¹⁵N. Isotopes of oxygen include ¹⁶O, ¹⁷O, and ¹⁸O.

### Alkyls

The terms “alkyl” and “substituted alkyl” are interchangeable and include substituted, optionally substituted and unsubstituted C₁-C₁₀ straight chain saturated aliphatic hydrocarbon groups, substituted, optionally substituted and unsubstituted C₂-C₁₀ straight chain unsaturated aliphatic hydrocarbon groups, substituted, optionally substituted and unsubstituted C₂-C₁₀ branched saturated aliphatic hydrocarbon groups, substituted and unsubstituted C₂-C₁₀ branched unsaturated aliphatic hydrocarbon groups, substituted, optionally substituted and unsubstituted C₃-C₈ cyclic saturated aliphatic hydrocarbon groups, substituted, optionally substituted and unsubstituted C₅-C₈ cyclic unsaturated aliphatic hydrocarbon groups having the specified number of carbon atoms. For example, the definition of “alkyl” shall include but is not limited to: methyl (Me), trideuteromethyl (—CD₃), ethyl (Et), propyl (Pr), butyl (Bu), pentyl, hexyl, heptyl, octyl, nonyl, decyl, undecyl, ethenyl, propenyl, butenyl, penentyl, hexenyl, heptenyl, octenyl, nonenyl, decenyl, undecenyl, 3-methylthiopropyl, 5-methylthiopentyl, 5-methylsulfinylpentane, methylsulfinyl)propane, 4-methylthiobutyl, 4-(Methanesulfonyl)butyl, 4-methylsulfinylbutane, isopropyl (i-Pr), isobutyl (t-Bu), tert-butyl (t-Bu), sec-butyl (s-Bu), isopentyl, neopentyl, cyclopropyl, cyclobutyl, cyclopentyl, cyclohexyl, cycloheptyl, cyclooctyl, cyclopentenyl, cyclohexenyl, cycloheptenyl, cyclooctenyl, methylcyclopropyl, ethylcyclohexenyl, butenylcyclopentyl, adamantyl, norbornyl and the like. Alkyl substituents are independently selected from the group consisting of hydrogen, halogen, —OH, —SH, —NH₂, —CN, —NO₂, ═O, ═CH₂, trihalomethyl, carbamoyl, arylC₀₋₁₀alkyl, heteroarylC0-10alkyl, C₁₋₁₀alkyloxy, arylC₀₋₁₀alkyloxy, C₁₋₁₀alkylthio, arylC₀₋₁₀alkylthio, C₁₋₁₀alkylamino, arylC₀₋₁₀alkylamino, N-aryl-N—C₀₋₁₀alkylamino, C₁₋₁₀alkylcarbonyl, arylC₀₋₁₀alkylcarbonyl, C₁₋₁₀alkylcarboxy, arylC₀₋₁₀alkylcarboxy, C₁₋₁₀alkylcarbonylamino, arylC₀₋₁₀alkylcarbonylamino, tetrahydrofuryl, morpholinyl, piperazinyl, hydroxypyronyl, —C₀₋₁₀alkylCOOR₂₁ and —C₀₋₁₀alkylCONR₂₂R₂₃ wherein R₂₁, R₂₂ and R₂₃ are independently selected from the group consisting of hydrogen, alkyl, allyl, aryl, or R₂₂ and R₂₃ are taken together with the nitrogen to which they are attached forming a saturated cyclic or unsaturated cyclic system containing 3 to 8 carbon atoms with at least one substituent as defined herein. Alternatively, aliphatic straight chain alcohols, aliphatic branched chain alcohols, aliphatic straight chain ketones or esters may be included in alkyls.

The term “saturated alkyl” means a straight-chain or branched-chain saturated alkyl which, unless otherwise specified, contains from about 1 to about 20 carbon atoms, preferably from about 1 to about 10 carbon atoms, more preferably from about 1 to about 8 carbon atoms, and most preferably from about 1 to about 6 carbon atoms. Examples of saturated alkyls include, but are not limited to, methyl, ethyl, propyl, isopropyl, n-butyl, sec-butyl, isobutyl, tert-butyl, pentyl, isoamyl, hexyl, octyl, dodecanyl, and the like. Saturated alkyl substituents can be unsubstituted or substituted, for example, with at least one substituent selected from the group consisting of a halogen, a nitro, an amino, a hydroxyl, a thio, an acyl, an alkyl, and a cyano.

The term “unsaturated alkyl” means saturated alkyl (straight-chain or branched-chain), as defined herein, in which one or more of the single carbon-carbon bonds thereof is instead a multiple bond, for example, a double or a triple bond. Thus, unsaturated alkyls include alkenyl and alkynyl substituents, as well as substituents that have a combination of double and triple bonds. The term “alkenyl” means a straight-chain or branched-chain alkenyl having one or more double bonds. Unless otherwise specified, the alkenyl can contain from about 2 to about 20 carbon atoms, preferably from about 2 to about 10 carbon atoms, more preferably from about 2 to about 8 carbon atoms, and most preferably from about 2 to about 6 carbon atoms. Examples of alkenyls include vinyl, allyl, 1,4-butadienyl, isopropenyl, and the like. The term “alkynyl” means a straight-chain or branched-chain alkynyl radical having one or more triple bonds. Unless otherwise specified, alkynyls can contain from about 2 to about 20 carbon atoms, preferably from about 2 to about 10 carbon atoms, more preferably from about 2 to about 8 carbon atoms, and most preferably from about 2 to about 6 carbon atoms. Examples of alkynyls include ethynyl, propynyl (propargyl), butynyl, and the like. Unsaturated alkyl substituents can be unsubstituted or substituted, for example, with at least one substituent selected from the group consisting of a halogen, a nitro, an amino, a hydroxyl, a thio, an acyl, an alkyl, and a cyano.

Unless otherwise specified, unsaturated alkyls, as defined herein, can contain from about 2 to about 20 carbon atoms, preferably from about 2 to about 10 carbon atoms, more preferably from about 2 to about 8 carbon atoms, and most preferably from about 2 to about 6 carbon atoms. Unsaturated alkyl substituents can be unsubstituted or substituted, for example, with at least one substituent selected from the group consisting of a halogen, a nitro, an amino, a hydroxyl, a thio, an acyl, an alkyl, and a cyano.

The term “alkylthio” (e.g. methylthio, ethylthio, propylthio, cyclohexenylthio and the like) represents a substituted or unsubstituted alkyl group as defined above having the indicated number of carbon atoms attached through a sulfur bridge. The term “alkylthioalkyl” represents an alkylthio group attached through an alkyl or substituted alkyl group as defined above having the indicated number of carbon atoms.

The term “alkylamino” (e.g. methylamino, diethylamino, butylamino, N-propyl-N-hexylamino, (2-cyclopentyl)propylamino, hexenylamino, and the like) represents one or two substituted or unsubstituted alkyl groups as defined above having the indicated number of carbon atoms attached through an amine bridge. The substituted or unsubstituted alkyl groups maybe taken together with the nitrogen to which they are attached forming a saturated cyclic or unsaturated cyclic system containing 3 to 10 carbon atoms with at least one substituent as defined above. The term “alkylaminoalkyl” represents an alkylamino group attached through a substituted or unsubstituted alkyl group as defined above having the indicated number of carbon atoms.

The term “alkylhydrazino” (e.g. methylhydrazino, diethylhydrazino, butylhydrazino, (2-cyclopentyl)propylhydrazino, cyclohexanehydrazino, and the like) represents one or two substituted or unsubstituted alkyl groups as defined above having the indicated number of carbon atoms attached through a nitrogen atom of a hydrazine bridge. The substituted or unsubstituted alkyl groups maybe taken together with the nitrogen to which they are attached forming a saturated cyclic or unsaturated cyclic system containing 3 to 10 carbon atoms with at least one substituent as defined above. The term “alkylhydrazinoalkyl” represents an alkylhydrazino group attached through a substituted or unsubstituted alkyl group as defined above having the indicated number of carbon atoms.

The term “alkylcarbonyl” (e.g. cyclooctylcarbonyl, pentylcarbonyl, 3-hexenylcarbonyl and the like) represents a substituted or unsubstituted alkyl group as defined above having the indicated number of carbon atoms attached through a carbonyl group. The term “alkylcarbonylalkyl” represents an alkylcarbonyl group attached through a substituted or unsubstituted alkyl group as defined above having the indicated number of carbon atoms.

The term “alkylcarboxy” (e.g. heptylcarboxy, cyclopropylcarboxy, 3-pentenylcarboxy and the like) represents an alkylcarbonyl group as defined above wherein the carbonyl is in turn attached through an oxygen bridge. The term “alkylcarboxyalkyl” represents an alkylcarboxy group attached through an alkyl group as defined above having the indicated number of carbon atoms.

The term “alkylcarbonylamino” (e.g. hexylcarbonylamino, cyclopentylcarbonyl-aminomethyl, methylcarbonylaminophenyl and the like) represents an alkylcarbonyl group as defined above wherein the carbonyl is in turn attached through the nitrogen atom of an amino group. The nitrogen group may itself be substituted with a substituted or unsubstituted alkyl or aryl group. The term “alkylcarbonylaminoalkyl” represents an alkylcarbonylamino group attached through a substituted or unsubstituted alkyl group as defined above having the indicated number of carbon atoms.

The term “alkylcarbonylhydrazino” (e.g. ethylcarbonylhydrazino, tert-butylcarbonylhydrazino and the like) represents an alkylcarbonyl group as defined above wherein the carbonyl is in turn attached through the nitrogen atom of a hydrazino group.

The term “cycloalkylcarbonyl”, means an acyl group derived from a monocyclic or bridged cycloalkanecarboxylic acid such as cyclopropanecarbonyl, cyclohexanecarbonyl, adamantanecarbonyl, and the like, or from a benz-fused monocyclic cycloalkanecarboxylic acid which is optionally substituted by, for example, alkylamino, such as 1,2,3,4-tetrahydro-2-naphthoyl, 2-acetamido-1,2,3,4-tetrahydro-2-naphthoyl.

The term “alkyloxy” (e.g. methoxy, ethoxy, propyloxy, allyloxy, cyclohexyloxy) represents a substituted or unsubstituted alkyl group as defined above having the indicated number of carbon atoms attached through an oxygen bridge. The term “alkyloxyalkyl” represents an alkyloxy group attached through an alkyl or substituted alkyl group as defined above having the indicated number of carbon atoms.

Methyl-thio-alkyl, such as CH₃S(CH₂)ₙ—X, wherein n is between 2 and 10. Methyl-sulfinyl-Alkyl, such as CH₃SO(CH₂)ₙ, wherein n is between 2 and 12. Alternatively, a methyl-sulfonyl-alkyl, such as CH₃SO₂(CH₂)ₙ—, where n is between 2 and 10. Alternatively, a thio-hydroxy-alkyl, a sufinyl-hydroxy-alkyl, or a sulfonylhydroxy-alkyl; thio-oxo-alkyl, sufinyl-oxo-alkyl, thio-alkene, sufinyl-alkene, sulfonyl-alkene, mercapto-alkyl, cysteine-thio-alkyl, disufanyl (either dimeric 4-mercaptobutyl or (4-glucopyranosyldisulfanyl)butyl.

Or “olefins” or “alkene”, which are any unsaturated hydrocarbon containing one or more pairs of carbon atoms linked by a double bond, with either (1) as cyclic or acyclic (aliphatic) olefins, in which the double bond is located between carbon atoms forming part of a cyclic (closed-ring) or of an open-chain grouping, respectively, and (2) as monoolefins, diolefins, triolefins, etc., in which the number of double bonds per molecule is, respectively, one, two, three, or some other number. Olefins may include branched alcohol groups or straight and branched chain configurations.

Aryls

The term “aryl”, alone or in combination, represents an unsubstituted, mono-, or polysubstituted monocyclic, polycyclic, biaryl aromatic groups covalently attached at any ring position capable of forming a stable covalent bond, certain preferred points of attachment being apparent to those skilled in the art (e.g., 3-phenyl, 4-naphthyl and the like) and means a phenyl or naphthyl radical which optionally carries one or more substituents selected from alkyl, alkoxy, halogen, hydroxy, amino and the like, such as phenyl, p-tolyl, 4-methoxyphenyl, 4-(tert-butoxy)phenyl, 4-fluorophenyl, 4-chlorophenyl, 4-hydroxyphenyl, 1-naphthyl, 2-naphthyl and the like, and phenyl, polyphenols, pentadeuterophenyl, biphenyl, naphthyl, dihydronaphthyl, tetrahydronaphthyl, indenyl, indanyl, azulenyl, anthryl, phenanthryl, fluorenyl, (methylsulfonyl)propane, pyrenyl and the like. The aryl substituents are independently selected from the group consisting of hydrogen, halogen, —OH, —SH, —CN, —NO₂, trihalomethyl, hydroxypyronyl, C₁₋₁₀alkyl, arylC₀₋₁₀alkyl, C₀₋₁₀alkyloxyC₀₋₁₀alkyl, arylC0-10alkyloxyC₀₋₁₀alkyl, Co₀₋₁₀alkylthioC₀₋₁₀alkyl, arylC₀₋₁₀alkylthioC₀₋₁₀alkyl, C₀₋₁₀alkylaminoC₀₋₁₀alkyl, arylC₀₋₁₀alkylaminoC₀₋₁₀alkyl, N-aryl-N—O₀₋₁₀alkylaminoC₀₋₁₀alkyl, C₀₋₁₀alkylcarbonylC₀₋₁₀alkyl, arylC₀₋₁₀alkylcarbonylC₀₋₁₀alkyl, C₁₋₁₀alkylcarboxyC₀₋₁₀alkyl, arylC₀₋₁₀alkylcarboxyC₀₋₁₀alkyl, C₁₋₁₀alkylcarbonylaminoC₀₋₁₀alkyl, arylC₀₋₁₀alkylcarbonylaminoC₀₋₁₀alkyl, —C₀₋₁₀alkylCOOR₂₁, and —C₀₋₁₀alkylCONR₂₂R₂₃ wherein R₂₁, R₂₂ and R₂₃ are independently selected from the group consisting of hydrogen, alkyl, aryl or R₂₂ and R₂₃ are taken together with the nitrogen to which they are attached forming a saturated cyclic or unsaturated cyclic system containing 3 to 8 carbon atoms with at least one substituent as defined above. The term “aryl” may also mean an aromatic carbocyclic radical, as commonly understood in the art, and includes monocyclic and polycyclic aromatics such as, for example, phenyl and naphthyl rings. Preferably, the aryl comprises one or more six-membered rings including, for example, phenyl, naphthyl, and biphenyl. Aryl substituents can be unsubstituted or substituted, for example, with at least one substituent selected from the group consisting of a halogen, a nitro, an amino, a hydroxyl, a thio, an acyl, an alkyl, and a cyano.

The term “arylalkoxy carbonyl”, alone or in combination, means a radical of the formula —C(O)—O-arylalkyl, in which the term “arylalkyl” has the significance given below. An example of an arylalkoxycarbonyl radical is benzyloxycarbonyl or benzoates.

The term “arylalkyl” means an alkyl radical in which one hydrogen atom is replaced by an aryl radical, such as benzyl, phenylethyl, phenpropyl, phenbutyl, and the like. The term “arylalkyl” (e.g. (4-hydroxyphenyl)ethyl, (2-aminonaphthyl)hexenyl and the like) represents an aryl group as defined above attached through a substituted or unsubstituted alkyl group as defined above having the indicated number of carbon atoms.

The term “arylcarbonyl” (e.g. 2-thiophenylcarbonyl, 3-methoxyanthrylcarbonyl and the like) represents an aryl group as defined above attached through a carbonyl group.

The term “arylalkylcarbonyl” (e.g. (2,3-dimethoxyphenyl)propylcarbonyl, (2-chloronaphthyl)pentenyl-carbonyl and the like) represents an arylalkyl group as defined above wherein the alkyl group is in turn attached through a carbonyl.

The term “aryloxy” (e.g. phenoxy, naphthoxy, 3-methylphenoxy, and the like) represents an aryl or substituted aryl group as defined above having the indicated number of carbon atoms attached through an oxygen bridge. The term “aryloxyalkyl” represents an aryloxy group attached through a substituted or unsubstituted alkyl group as defined above having the indicated number of carbon atoms.

The term “aryloxycarbonyl” (e.g. phenoxycarbonyl, naphthoxycarbonyl) represents a substituted or unsubstituted aryloxy group as defined above having the indicated number of carbon atoms attached through a carbonyl bridge.

The term “arylthio” (e.g. phenylthio, naphthylthio, 3-bromophenylthio, and the like) represents an aryl or substituted aryl group as defined above having the indicated number of carbon atoms attached through a sulfur bridge. The term “arylthioalkyl” represents an arylthio group attached through a substituted or unsubstituted alkyl group as defined above having the indicated number of carbon atoms.

The term “arylamino” (e.g. phenylamino, diphenylamino, naphthylamino, N-phenyl-N-naphthylamino, o-methylphenylamino, p-methoxyphenylamino, and the like) represents one or two aryl groups as defined above having the indicated number of carbon atoms attached through an amine bridge. The term “arylaminoalkyl” represents an arylamino group attached through a substituted or unsubstituted alkyl group as defined above having the indicated number of carbon atoms. The term “arylalkylamino” represents an aryl group attached through an alkylamino group as defined above having the indicated number of carbon atoms. The term “N-aryl-N-alkylamino” (e.g. N-phenyl-N-methylamino, N-naphthyl-N-butylamino, and the like) represents one aryl and one a substituted or unsubstituted alkyl group as defined above having the indicated number of carbon atoms independently attached through an amine bridge.

The term “arylhydrazino” (e.g. phenylhydrazino, naphthylhydrazino, 4-methoxyphenylhydrazino, and the like) represents one or two aryl groups as defined above having the indicated number of carbon atoms attached through a hydrazine bridge. The term “arylhydrazinoalkyl” represents an arylhydrazino group attached through a substituted or unsubstituted alkyl group as defined above having the indicated number of carbon atoms. The term “arylalkylhydrazino” represents an aryl group attached through an alkylhydrazino group as defined above having the indicated number of carbon atoms. The term “N-aryl-N-alkylhydrazino” (e.g. N-phenyl-N-methylhydrazino, N-naphthyl-N-butylhydrazino, and the like) represents one aryl and one a substituted or unsubstituted alkyl group as defined above having the indicated number of carbon atoms independently attached through an amine atom of a hydrazine bridge.

The term “arylcarboxy” (e.g. phenylcarboxy, naphthylcarboxy, 3-fluorophenylcarboxy and the like) represents an arylcarbonyl group as defined above wherein the carbonyl is in turn attached through an oxygen bridge. The term “arylcarboxyalkyl” represents an arylcarboxy group attached through a substituted or unsubstituted alkyl group as defined above having the indicated number of carbon atoms.

The term “arylcarbonylamino” (e.g. phenylcarbonylamino, naphthylcarbonylamino, 2-methylphenylcarbonylamino and the like) represents an arylcarbonyl group as defined above wherein the carbonyl is in turn attached through the nitrogen atom of an amino group. The nitrogen group may itself be substituted with a substituted or unsubstituted alkyl or aryl group.

The term “arylcarbonylaminoalkyl” represents an arylcarbonylamino group attached through a substituted or unsubstituted alkyl group as defined above having the indicated number of carbon atoms. The Nitrogen group may itself be substituted with a substituted or unsubstituted alkyl or aryl group.

The term “arylcarbonylhydrazino” (e.g. phenylcarbonylhydrazino, naphthylcarbonylhydrazino, and the like) represents an arylcarbonyl group as defined above wherein the carbonyl is in turn attached through the Nitrogen atom of a hydrazino group.

The term “arylalkanoyl” means an acyl radical derived from an aryl-substituted alkanecarboxylic acid such as phenylacetyl, 3-phenylpropionyl, hydrocinnamoyl, 4-phenylbutyryl, (2-naphthyl)acetyl, 4-chlorohydrocinnamoyl, 4-aminohydrocinnamoyl, 4-methoxyhydrocinnamoyl, and the like.

The term “aroyl” means an acyl radical derived from an aromatic carboxylic acid. Examples of such radicals include aromatic carboxylic acid, an optionally substituted benzoic or naphthoic acids such as benzoyl, 4-chlorobenzoyl, 4-carboxybenzoyl, 4-[(benzyloxy)carbonyl]benzoyl, 1-naphthoyl, 2-naphthoyl, 6-carboxy-2-naphthoyl, 6-[(benzyloxy) carbonyl]-2-naphthoyl, 3-benzyloxy-2-naphthoyl, 3-hydroxy-2-naphthoyl, 3-[(benzyloxy)formamido]-2-naphthoyl, and the like.

The term “aryloxyalkanoyl” means an acyl radical of the formula aryl-O-alkanoyl.

The term “polyphenols” is characterized by the presence of more than one phenol unit or building block per molecule, including hydrolyzable tannins (gallic acid esters of glucose and other sugars) and phenylpropanoids, such as lignins, flavonoids, and condensed tannins. The —OH groups in the polyphenols will conjugate with the pseudohalides. One example of a polyphenol is curcumin, which is (1,7-bis(4-hydroxy-3-methoxyphenyl)-1,6-heptadiene-3,5-dione), and is the principal curcuminoid of the popular Indian spice turmeric, which is a member of the ginger family (Zingiberaceae). Another polyphenol is sulforaphane, sulfoaphene, erysolin, erucin, iberin, alyssin, berteroin, iberverin, cheirolin

Heteros

The term “heterocyclyl”, as used herein except where noted, represents a stable 5- to 7-membered mono or bicyclic or stable 7- to 10-membered bicyclic heterocyclic ring which is either saturated or unsaturated, and which consists of carbon atoms, and from one to three heteroatoms selected from the group consisting of N, O, S, and wherein the nitrogen and sulfur heteroatoms may be optionally oxidized, and the nitrogen atom may optionally be quaternized, and including any bicyclic group in which any of the above defined heterocyclic rings is fused to a benzene ring. The heterocyclic ring may be attached at any heteroatom or carbon atom which results in the creation of a stable structure. Examples of such heterocyclic elements, commonly known as heterocyclyl include piperidinyl, piperazinyl, 2-oxopiperazinyl, 2-oxopiperazinyl, 2-oxopiperidinyl, 2-oxopyrrolidinyl, 2-oxoazepinyl, azepinyl, pyrrolyl, 4-piperidonyl, pyrrolidinyl, pyrazolyl, pyrazolidinyl, imidazolyl, imidazolinyl, imidazolidinyl, pyridyl, pyrazinyl, pyrimidinyl, pyridazinyl, oxazolidinyl, isoxazolyl, isoxazolidinyl, morpholinyl, thiazolyl, thiazolidinyl, isothiazolyl, quinuclidinyl, isothiazolidinyl, indolyl, quinolinyl, isoquinolinyl, benzimidazolyl, thiadiazolyl, benzopyranyl, benzothiazolyl, benzoxazolyl, furyl, tetrahydrofuryl, tetrahydropyranyl, thienyl, benzothienyl, indoles, tetrahydroquinolinyl (e.g. 1,2,3,4-tetrahydro-2-quinolinyl, etc.), 1,2,3,4-tetrahydro-isoquinolinyl (e.g. 1,2,3,4-tetrahydro-1-oxo-isoquinolinyl etc.), quinoxalinyl, beta-carbolinyl, 2-benzofurancarbonyl, thiamorpholinyl, thiamorpholinyl sulfoxide, thiamorpholinyl sulfone, oxadiazolyl, polycyclic aromatic hydrocarbons, and the like. The heterocycle may be substituted in a manner which results in the creation of a stable structure.

The terms “heteroaryl”, “heterocycle” or “heterocyclic” refers to a monovalent unsaturated group having a single ring or multiple condensed rings, from 1 to 13 carbon atoms and from 1 to 10 hetero atoms selected from the group consisting of nitrogen, sulfur, and oxygen, within the ring. The heteroaryl groups in this invention can be optionally substituted with 1 to 10 substituents selected from the group consisting of: hydrogen, halogen, —OH, —SH, —CN, —NO₂, trihalomethyl, hydroxypyronyl, C₁₋₁₀alkyl, arylC₀₋₁₀alkyl, C₀₋₁₀alkyloxyC₀₋₁₀alkyl, arylC0-10alkyloxyC₀₋₁₀alkyl, C₀₋₁₀alkylthioC₀₋₁₀alkyl, arylC₀₋₁₀alkylthioC₀₋₁₀alkyl, C₀₋₁₀alkylaminoC₀₋₁₀alkyl, arylC₀₋₁₀alkylaminoC₀₋₁₀alkyl, N-aryl-N—C₀₋₁₀alkylaminoC₀₋₁₀alkyl, C₁₋₁₀alkylcarbonylC₀₋₁₀alkyl, arylC₀₋₁₀alkylcarbonylC₀₋₁₀alkyl, C₁₋₁₀alkylcarboxyC₀₋₁₀alkyl, arylC₀₋₁₀alkylcarboxyC₀₋₁₀alkyl, C₁₋₁₀alkylcarbonylaminoC₀₋₁₀alkyl, arylC₀₋₁₀alkylcarbonylaminoC₀₋₁₀alkyl, —Co₀₋₁₀alkylCOOR₂₁, and —C₀₋₁₀alkylCONR₂₂R₂₃ wherein R₂₁, R₂₂ and R₂₃ are independently selected from the group consisting of hydrogen, alkyl, aryl or R₂₂ and R₂₃ are taken together with the nitrogen to which they are attached forming a saturated cyclic or unsaturated cyclic system containing 3 to 8 carbon atoms with at least one substituent as defined above.

The definition of “heteroaryl” includes but is not limited to thienyl, benzothienyl, isobenzothienyl, 2,3-dihydrobenzothienyl, furyl, pyranyl, benzofuranyl, isobenzofuranyl, 2,3-dihydrobenzofuranyl, pyrrolyl, pyrrolyl-2,5-dione, 3-pyrrolinyl, indolyl, isoindolyl, 3H-indolyl, indolinyl, indolizinyl, indazolyl, phthalimidyl (or isoindoly-1,3-dione), imidazolyl, 2H-imidazolinyl, benzimidazolyl, deuterobenzimidazolyl, dideuterobenzimidazolyl, trideuterobenzimidazolyl, tetradeuterobenzimidazolyl, pyridyl, deuteropyridyl, dideuteropyridyl, trideuteropyridyl, tetradeuteropyridyl, pyrazinyl, pyradazinyl, pyrimidinyl, triazinyl, quinolyl, isoquinolyl, 4H-quinolizinyl, cinnolinyl, phthalazinyl, quinazolinyl, quinoxalinyl, 1,8-naphthyridinyl, pteridinyl, carbazolyl, acridinyl, phenazinyl, phenothiazinyl, phenoxazinyl, chromanyl, benzodioxolyl, piperonyl, purinyl, pyrazolyl, triazolyl, tetrazolyl, thiazolyl, isothiazolyl, benzthiazolyl, oxazolyl, isoxazolyl, benzoxazolyl, oxadiazolyl, thiadiazolyl, pyrrolidinyl-2,5-dione, imidazolidinyl-2,4-dione, 2-thioxo-imidazolidinyl-4-one, imidazolidinyl-2,4-dithione, thiazolidinyl-2,4-dione, 4-thioxo-thiazolidinyl-2-one, piperazinyl-2,5-dione, tetrahydro-pyridazinyl-3,6-dione, 1,2-dihydro-[1,2,4,5]tetrazinyl-3,6-dione, [1,2,4,5]tetrazinanyl-3,6-dione, dihydro-pyrimidinyl-2,4-dione, pyrimidinyl-2,4,6-trione, 1H-pyrimidinyl-2,4-dione, 5-iodo-1H-pyrimidinyl-2,4-dione, 5-chloro-1H-pyrimidinyl-2,4-dione, 5-methyl-1H-pyrimidinyl-2,4-dione, 5-isopropyl-1H-pyrimidinyl-2,4-dione, 5-propynyl-1H-pyrimidinyl-2,4-dione, 5-trifluoromethyl-1H-pyrimidinyl-2,4-dione, 6-amino-9H-purinyl, 2-amino-9H-purinyl, 4-amino-1H-pyrimidinyl-2-one, 4-amino-5-fluoro-1H-pyrimidinyl-2-one, 4-amino-5-methyl-1H-pyrimidinyl-2-one, 2-amino-1,9-dihydro-purinyl-6-one, 1,9-dihydro-purinyl-6-one, 1H-[1,2,4]triazolyl-3-carboxylic acid amide, 2,6-diamino-N-6-cyclopropyl-9H-purinyl, 2-amino-6-(4-methoxyphenylsulfanyl)-9H-purinyl, 5,6-dichloro-1H-benzoimidazolyl, 2-isopropylamino-5,6-dichloro-1H-benzoimidazolyl, 2-bromo-5,6-dichloro-1H-benzoimidazolyl, 5-methoxy-1H-benzoimidazolyl, 3-ethylpyridyl, 5-methyl-2-phenyl-oxazolyl, 5-methyl-2-thiophen-2-yl-oxazolyl, 2-furan-2-yl-5-methyl-oxazolyl, 3-methyl-3H-quinazolin-4-one, 4-methyl-2H-phthalazin-1-one, 2-ethyl-6-methyl-3H-pyrimidin-4-one, 5-methoxy-3-methyl-3H-imidazo[4,5-b]pyridine and the like. For the purposes of this application, the terms “heteroaryl”, “heterocycle” or “heterocyclic” do not include carbohydrate rings (i.e. mono- or oligosaccharides).

The term “saturated heterocyclic” represents an unsubstituted, mono-, and polysubstituted monocyclic, polycyclic saturated heterocyclic group covalently attached at any ring position capable of forming a stable covalent bond, certain preferred points of attachment being apparent to those skilled in the art (e.g., 1-piperidinyl, 4-piperazinyl, DBU, and the like). The saturated heterocyclic substituents are independently selected from the group consisting of halo, —OH, —SH, —CN, —NO.₂, trihalomethyl, hydroxypyronyl, C₁₋₁₀alkyl, arylC₀₋₁₀alkyl, C₀₋₁₀alkyloxyC₀₋₁₀alkyl, arylC₀₋₁₀alkyloxyC₀₋₁₀alkyl, C₀₋₁₀alkylthioC₀₋₁₀alkyl, arylC₀₋₁₀alkylthioC₀₋₁₀alkyl, C₀₋₁₀alkylaminoC₀₋₁₀alkyl, arylC₀₋₁₀alkylaminoC₀₋₁₀alkyl, N-aryl-N—C₀₋₁₀alkylaminoC₀₋₁₀alkyl, C₁₋₁₀alkylcarbonylC₀₋₁₀alkyl, arylC₀₋₁₀alkylcarbonylC₀₋₁₀alkyl, C₁₋₁₀alkylcarboxyC₀₋₁₀alkyl, C₀₋₁₀alkylcarboxyC₀₋₁₀alkyl, C₁₋₁₀alkylcarbonylaminoC₀₋₁₀alkyl, arylC₀₋₁₀alkylcarbonylaminoC₀₋₁₀alkyl, —C₀₋₁₀alkylCOOR₂₁, and —C₀₋₁₀alkylCONR₂₂R₂₃ wherein R₂₁, R₂₂ and R₂₃ are independently selected from the group consisting of hydrogen, alkyl, aryl or R₂₂ and R₂₃ are taken together with the nitrogen to which they are attached forming a saturated cyclic or unsaturated cyclic system containing 3 to 8 carbon atoms with at least one substituent as defined above.

The definition of saturated heterocyclic includes, but is not limited to, pyrrolidinyl, pyrazolidinyl, piperidinyl, 1,4-dioxanyl, morpholinyl, 1,4-dithienyl, thiomorpholinyl, piperazinyl, quinuclidinyl, and the like.

The term “heterocyclyloxycarbonyl”, means an acyl group derived from heterocyclyl-O—CO— wherein heterocyclyl is defined above.

The term “heterocyclylalkanoyl” is an acyl radical derived from a heterocyclyl-substituted alkane carboxylic acid wherein heterocyclyl has the same significance given above.

The term “heterocyclylalkoxycarbonyl” means an acyl radical derived from a heterocyclyl-substituted alkyl-O—COOH wherein heterocyclyl has the same significance given above.

The term “polycyclic aromatic hydrocarbons” are fused aromatic rings and do not contain heteroatoms or carry substituents. One example of a polycyclic aromatic hydrocarbon is antracene, which consists of three fused benzene rings.

The term “naphthalene”, also known as naphthalin, is bicyclo[4.4.0]deca-1,3,5,7,9-pentene or antimite is a crystalline, aromatic, white, solid hydrocarbon with formula C₁₀H₈ and the structure of two fused benzene rings.

Fatty Acids

The term “fatty acid” means a carboxylic acid with a long unbranched aliphatic tail (chain), which is either saturated or unsaturated. Most naturally occurring fatty acids have a chain of four to 28 carbons.

The term “lipids” means fats, waxes, sterols, fat-soluble vitamins (such as vitamins A, D, E and K), monoglycerides, diglycerides, phospholipids, and others that are hydrophobic or amphiphilic small molecules; where biological lipids originate entirely or in part from two distinct types of biochemical subunits or “building blocks”: ketoacyl and isoprene groups, including fatty acyls, glycerolipids, glycerophospholipids, sphingolipids, saccharolipids and polyketides. Glycerolipids are composed mainly of mono-, di- and tri-substituted glycerols, which are fatty-acid esters of glycerol (triacylglycerols), also known as triglycerides. Glycerophospholipids, also referred to as phospholipids, include phosphatidylcholine (also known as PC, GPCho or lecithin), phosphatidylethanolamine (PE or GPEtn) and phosphatidylserine (PS or GPSer), phosphatidylinositols and phosphatidic acids, Sphingolipids, Sterol lipids, such as cholesterol and its derivatives. Prenol lipids are synthesized from the 5-carbon precursors isopentenyl diphosphate and dimethylallyl diphosphate. Saccharolipids are fatty acids which are linked directly to a sugar backbone.

The term “oils” means any substance that is liquid at ambient temperatures and is hydrophobic but soluble in organic solvents that has a high carbon and hydrogen content and are nonpolar substances. Oils include compound classes including vegetable oils, organic oils, and the like.

Amino Acids

“Amino acid residues” means any of the naturally occurring alpha-, beta-, and gamma-amino carboxylic acids; including their D and L optical isomers and racemic mixtures thereof, and the N-lower alkyl- and N-phenyl lower alkyl-derivatives of these amino acids. The amino acid residue is bonded through a nitrogen of the amino acid. The naturally occurring amino acids which can be incorporated into the present invention include, but are not limited to, alanine, arginine, asparagine, aspartic acid, cysteine, cystine, glutamic acid, glutamine, glycine, histidine, isoleucine, leucine, lysine, methionine, ornithine, phenylalanine, proline, serine, threonine, thyroxine, tryptophan, tyrosine, valine, beta-alanine, and gamma-aminobutyric acid. Preferred amino acid residues include proline, leucine, phenylalanine, isoleucine, alanine, .gamma.-amino butyric acid, valine, glycine, and phenylglycine.

All alpha-amino acids except glycine contain at least one asymmetric carbon atom. As a result, they are optically active, existing in either D or L form as a racemic mixture. Accordingly, some of the compounds of the present invention may be prepared in optically active form, or as racemic mixtures of the compounds claimed herein.

The term “A” wherein A is an amino acid or peptide of 2 to 3 amino acid residues refers to an amino acid or a peptide diradical starting with the HN radical on the left hand side of A and terminated by the —C(O) radical on the right hand side. For example, the amino acid glycine is abbreviated HAOH wherein A is HN—CH₂—C(O).

The term “aminoalkanoyl” means an acyl radical derived from an amino-substituted alkanecarboxylic acid wherein the amino group can be a primary, secondary or tertiary amino group containing substituents selected from hydrogen, and alkyl, aryl, arylalkyl, cycloalkyl, cycloalkylalkyl radicals and the like.

The term “glycoside” means a substituent that yields on hydrolysis a sugar, for example glucose, galactose, amino sugars (e.g., D-glucosamine and N-acetyl-.beta.-glucosamine), and the like.

Carbohydrates

“Carbohydrates” refers to any compound with the empirical formula Cₘ(H₂O)ₙ; that is, consists only of carbon, hydrogen and oxygen, with the hydrogen and oxygen in a 1:2:1 atom ratio (as in water), including, but not limited to, thioglucose, monosaccharides, disaccharides, oligosaccharides, polysaccharides, glycosylated carbohydrates, benzyl glycosides, cinnamic glycosides, selenos and the like.

Glycosylated carbohydrates include the following: glucoraphenin, glucocheirolin, Glucocapparin, Sinigrin, Glucoibervirin, Glucoiberin, Glucocheirolin, Glucoputranjivin, Glucosisymbrin, Glucoerysimumhieracifolium, Gluconapin, Progoitrin, Epiprogoitrin, Glucoerucin, Glucoraphasatin, Glucoraphanin, Glucoraphenin, Glucoarabidopsithalianain, Glucoconringiin, Glucoalyssin, Glucobrassicanapin, Gluconapoleiferin, Glucocleomin, Glucolesquerellin, Glucohesperin, Glucoarabishirsutain, Glucoarabishirsuin, Glucohirsutin, Glucobrassicin, 4-Hydroxyglucobrassicin, 4-Methoxyglucobrassicin, Neoglucobrassicin, Glucotropaeolin, Glucosinalbin, Gluconasturtiin, Glucobarbarin, Glucomalcomiin, and the like.

Salts

“Pharmaceutically acceptable, non-toxic salts” refers to pharmaceutically acceptable salts of the compounds of this invention which retain the biological activity of the parent compounds and are not biologically or otherwise undesirable (e.g. the salts are stable). Salts of the two types may be formed from the compounds of this invention: (1) salts of inorganic and organic bases from compounds of Formula I which have a carboxylic acid functional group. (2) Acid addition salts may be formed at the amine functional group of many of the compounds of this invention.

Pharmaceutically acceptable salts derived from inorganic bases include sodium, potassium, lithium, ammonium, calcium, magnesium, ferrous, zinc, copper, manganous, aluminum, ferric, manganic salts and the like. Particularly preferred are the ammonium, potassium, sodium, calcium and magnesium salts. Pharmaceutically acceptable, non-toxic salts derived from organic bases include salts of primary, secondary and tertiary amines, substituted amines including naturally occurring substituted amines, cyclic amines and basic ion exchange resins. Such salts are exemplified by, for example, isopropopylamine, trimethylamine, diethylamine, triethylamine, tripropylamine, ethanolamine, 2-dimethylaminoethanol, tromethamine, dicyclohexamine, lysine, arginine, histidine, caffeine, procaine, hydrabramine, choline, betaine, ethylenediamine, glucosamine, methylglucamine, theobromine, purines, piperazine, piperidine, N-ethylpiperidine, polyamine resins and the like. Particularly preferred organic non-toxic bases are isopropylamine, diethylamine, ethanolamine, piperidine, tromethamine, dicyclohexylamine, choline and caffeine.

The compounds of the present invention can be obtained by one of ordinary skill in the art by isolation from natural sources; chemical synthesis using well-known and readily available chemical reactions, reagents, and procedures; by semisynthesis; or the like.

The compounds limiting the ATPase function (uncoupler) and compositions of the present invention can be used medically to regulate biological phenomena including, but not limited to: acid production in the stomach, intra-organellar acidification of intracellular organelles; urinary acidification; bone resorption; fertility; angiogenesis; cellular invasiveness (e.g., tumor cell invasiveness); metastasis; and the development of drug resistance in tumor cells. Thus, the compounds of the present invention are useful in the treatment of diseases which can be controlled by the inhibition of ATPase. Such diseases include, for example, but are not limited to, cancer, osteoporosis, Alzheimer's disease, glaucoma, and abnormal urinary acidification. Moreover, the ATPase inhibitors of the present invention can be used in the treatment or prevention of diseases which utilize an acid-promoted cell penetration mechanism. For example, the compounds of the present invention can be used to inhibit the entry of viruses (e.g., baculoviruses and retroviruses), or to inhibit the entry of protein toxins (e.g., diphtheria toxin), into cells. The compounds of the present invention also can be used to inhibit fertility in an animal, for example, a human, or to inhibit the invasiveness or metastasis of tumor cells, or to promote the sensitivity of cancer toward drugs by inhibiting the ability of cancer cells to develop resistance to drugs, thereby facilitating and/or making possible the chemotherapeutic treatment of cancer.

Mode of Action

Generally speaking, isothiocyanates and thiocyanates, as designated by Formula I and 2, are compounds limiting the ATPase function (uncoupler) that produce an anti-secretory function for stomach acid and anti-mitotic action on stomach cancer. The traditional explanation by Post-Albers (PA) model to explain the molecular mechanism of action of H, K-ATPase system does not predict the antisecretory benefits of isothiocyanates and thiocyanates. However, a new Ray model 100 shows the clinical benefits of isothiocyanates and thiocyanates by uncoupling the H, K-ATPase system and thereby inhibiting the ion transport process.

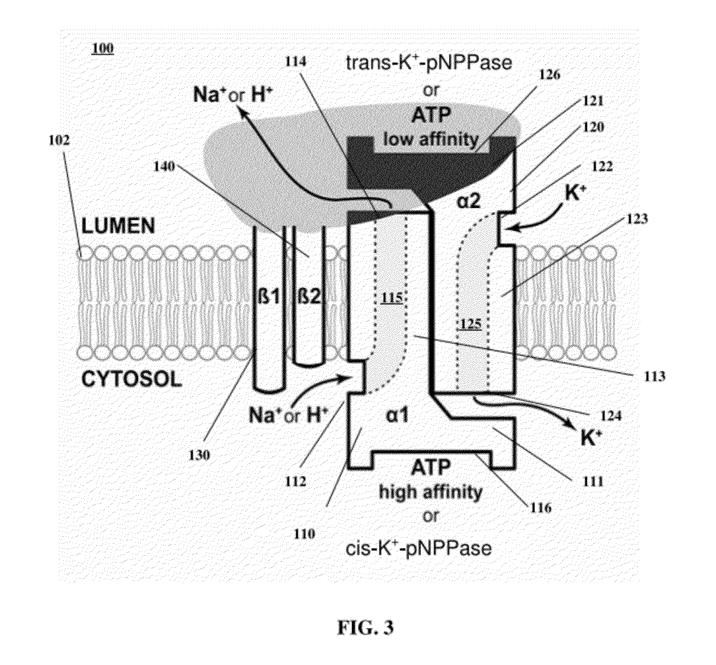

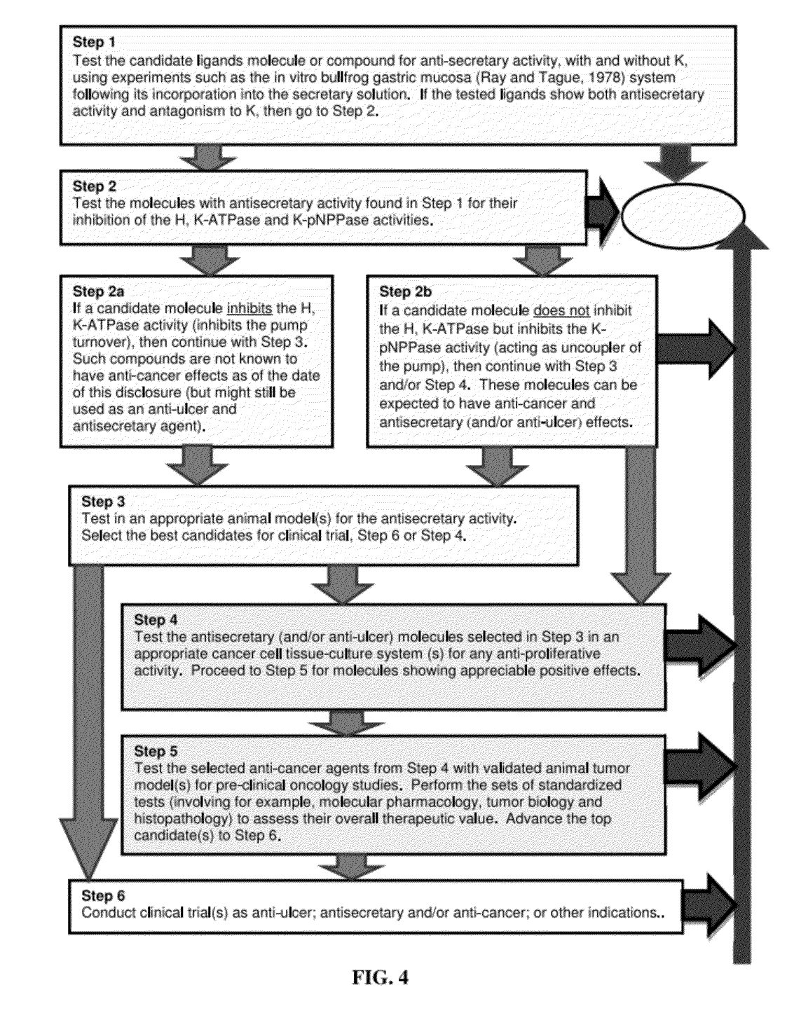

The PA model relies on only one alpha (α) subunit in the functional enzyme (single topology) that has one interchanging (to and fro) ion binding site to conduct the H (or Na) and K transport across the membrane. To the contrary, the Ray model 100 (given below) comprises a first alpha (α1) subunit 110 and a second alpha (α2) subunit 120, wherein the first and second alpha (α1 and α2) subunits 110 and 120 include a dual topology having opposing orientation (like mirror-image) across a membrane 102. The α1 subunit 110 includes a first head 111 and a first tail 113, whereby the first head 111 is included on the cytosol of the membrane 102 and the first tail 113 trans across the membrane 102. The α2 subunit 120 includes a second head 121 and a second tail 123, whereby the second head 121 is included on the lumen side of membrane 102 and the second tail 123 disposed across the membrane 102. The dual topology having an opposing orientation of the α1 and α2 subunits includes the first head 111 at or near the end of the second tail 123 and the second head at or near the end of the first tail 113, whereby the first tail 113 and the second tail 123 are substantially adjacent to each other across the membrane 102. The first alpha subunit 110 includes an ion channel 115 and the second alpha subunit includes a second ion channel 125, where the first alpha subunit 110 has a first and a second binding sites for H (or Na) 112 and 114 on the cytosol and lumen ends of the first ion channel 111 and the second alpha subunit 120 has a first and second binding sites for K transport 122 and 124 on the lumen and cytosol ends of the second ion channel 121, respectively, for simultaneous bidirectional translocation of ions across the membrane 102. In addition, the Ray model 100 includes a first high-affinity site 122 and second low affinity K site 124 at or near the respective ion-channels (as revealed by the manifestation of K-stimulated pNPPase activity) that are essential for the ion-channel function. Interference with this K site (essential for the trans-pNPPase activity as shown in FIG. 3)) by compounds such as thiocyanate, and isothiocyanate, and their derivatives uncouple the system thus inhibiting the ion translocation process. The PA model can not explain the observed antagonism between K and thiocyanate in gastric H transport. There is also a low-affinity ATP site 126 accessible from the cell exterior acting as the regulator of the ion channel function, as shown in FIG. 3. Homologues and analogues of ATP could be synthesized that interfere with this low-affinity ATP site inhibiting the ion transport.

As shown in FIG. 3, the new Ray model 100 shows a first beta (β1) subunit 130 and a second beta (β2) subunit 140, which are exposed to the cell surface exterior (lumen side) as part of the functional H, K-ATPase (αβ)2 complex. According to the newly revised model 100, as shown in FIG. 3, the two adjoining trans-bilayer α helix segments (α1 and α2) 110 and 120 that are held in close contact having opposite (up-down) orientation and experience the most perturbation during the enzymatic reaction causing ion transport.

Two identical catalytic subunits, α1 and α2 (about 100 k. Da each), are in intimate communion facing up and down respectively across the membrane bilayer 102 to form a α1α2 assembly with relevant embedded-ion channel in close contact. The α1α2 assembly is held together along with two closely associated first and second beta (β1 and β2) subunits 130 and 140 facing the lumen. In view of the recent cross-linking evidence (Ivanov, A V; Modyanov, N N and Askari, A. (2002), “Role of self-association of beta-subunits in the oligomeric structure of Na, K-ATPase”, Biochem J. 364, 293-299, incorporated by reference herein) of a direct β-β linkage in the native state of β1 and β2, the β1 and β2 must be held closely together with the α1α2 assembly. A cytosolic ATP hydrolytic site 116 (separate from the cis-pNPPase site at or near the ion channel) on the α1, and a trans-cytosolic (luminal) low affinity (non-hydrolysable) ATP-binding site 126 and corresponding trans-pNPPase site on the α2 are shown in FIG. 3. The high-affinity K site 122 responsible for the K-ATPase activity is located across the bilayer on α2 (designated by a half square at the cell exterior) and the corresponding high affinity H or Na site 112 (designated also by halfsquare in the cell interior) is on the cytosolic side of the α1; both being present in a relatively hydrophilic environment.

The low-affinity ion-binding sites 114 and 124 responsible for the release of the transported ions from respective channels are present at or near the related channel of the α-domain on each side of the bilayer. These low affinity sites 114 and 124 are responsible for the observed K-pNPPase reactions related to the ion channel activity. Appropriate cooperative interactions between the two α subunits 110 and 120 are essential for the ATPase mediated active cation transport although either the α1 or the α2 subunit can independently manifest the K-pNPPase reaction and the ATP binding process.

With the Ray Model 100, each α-subunit 110 and 120 holds its own built-in lipid-embedded ion channel 115 and 125, respectively, made up of the corresponding transmembrane helixes arranged along the channel periphery that carry H (or Na) and K respectively in opposite direction across the bilayer. The process of active ion transport is mediated by a series of intra- and inter-subunit events within the ATPase complex that involve binding of ATP, binding of H (or Na) and K, resultant phosphorylation, K induced dephosphorylation, and simultaneous release of cations (H or Na) due to the regeneration of E. The activity of K-pNPPase appears unrelated to the ATPase reaction, yet somehow related to the ion channel function (Ray, T K and Nandi J (1986) K-stimulated pNPPase is not a partial reaction of the gastric (H, K)-transporting ATPase: Evidence supporting a new model for the univalent cation transporting ATPase systems, Biochem. J. 233, 231-238, incorporated by reference herein).

Binding of high-affinity K to the designated α2-K-site 122 across the membrane bilayer releases the proton produced from ATP hydrolysis mediated by the enzyme's phosphorylated intermediates (E-P) and, in turn mediates the conformation-driven ion translocation. The conformational tension that is created by E-P within the lipid-embedded helix-domain of α1, in turn, induce changes in the adjoining (α2) helixes due to the strong hydrophobic force holding them together at the helix′-helix and lipid-helix interface where specific annular PC molecules supporting the ATPase function plays a significant role. (Sen, P. C. and Ray, T. K., (1980) “Control of K-stimulated ATPase of pig gastric microsomes: Effects of lipid environment and the endogenous activator”, Arch of Biochem. Biophysics. 202, 8-17; Nandi, J., Wright, M. V. and Ray, T. K. (1983), “Effects of phospholipase A2 on gastric microsomal H, K-ATPase system: Role of boundary lipids and the endogenous activator protein”, Biochemistry 22, 5814-5821; Ray et al, 2008, all incorporated by reference herein) Concurrently, the binding of high-affinity K to its specific site 122 on α2 provides the free energy needed to release the existing molecular (E-P) tension with simultaneous dephosphorylation of the intermediate, thus making the energy available for the H and K ion-transport across the membrane.

The low-affinity ATP binding site 126 on α2 within the lumen is analogous to the high-affinity ATP hydrolytic (α1) site 116 in the cytosol that has been changed in nature due to the steric influence of the overlaying glycoproteins (β-subunits) and now acts as a channel facilitator for H (or Na). The densely superimposed glycoprotein on the luminal α2 domain limits the access of ATP to its binding site, manifesting the apparent lower affinity. The heavily glycosylated β, in addition to its usual surface-recognition and other important roles in the cells also act as a buffering coat against acid damage to the lumenally exposed α2 residues in the parietal cells (Thangaraja, H., Wong, A., Chow, D. C., Crothers, J. M., and Forte, J. G. (2002) “Gastric H, K-ATPase and acid-resistant surface proteins”, Am J. Physiol. 282, G953-G961, incorporated by reference herein).

The site showing H or Na 112 and 114 implies that such model also fits the Na, K-ATPase system if the intracellular ionic environment demands such a change (Ray, T K and Nandi, J (1985), “Modulation of gastric H, K-transporting ATPase function by Sodium FEBS” Letters 185, 24-28, incorporated by reference herein). The α-subunit of both the gastric H, K-ATPase and Na, K-ATPase is a polypeptide (having 95-110 k. Da mass) consisting of about 1000 amino acids that shows remarkable (63%) sequence homology sharing many conserved domains (Blanco, G and Mercer, R W (1998), “Isozymes of the Na, K-ATPase: Heterogeneity in structure, diversity in function”, 275, F633-650; Lingrel, J B, Orlowski, J; Schull, M M and Price, E M 1990), “Molecular genetics of Na, K-ATPase”, Progress in Nuclic acid research and molecular Biology Vol 38, 39-83, all incorporated by reference herein).

The Ray dual-topology model 100 is a radical departure from the aforementioned PA model) in the following aspects: First, the Ray model 100 demands two alpha (2α) of reverse orientation (like mirror image) across the bilayer having two distinct ATP binding sites 116 and 126 (high and low affinity) along with two separate cation (H, Na or K) binding sites 112 and 120 across the bilayer for simultaneous transport of Na (or H) and K. Second, there are two distinct transmembrane ion channels 115 and 125, one within each alpha (α) subunit, responsible for the simultaneous transport of ions such as Na or H (in) and K (out) across the membrane 102. Third, the kinase and phosphatase steps are not separate steps in the Ray model 100, as they are simultaneously executed in unity (with simultaneous binding and release of ions across) during the vectorial translocation of ions.

It may be noted that unlike the simultaneous ion transport in the Ray model 100, the single topology PA model considers a sequential transport of ions involving only one catalytic (α) subunit undergoing periodic conformational shift. Lastly, there are two beta (2β) subunits for the functional ATPase complex in the Ray model 100 (instead of one beta β in the PA) that has multifarious interaction with the alpha subunit at the cell exterior giving structural and functional integrity to the alpha in addition to providing other roles.

Based on the dual-topology model, as shown in FIG. 3, there are several novel ways that the activity and function of the Na, K-ATPase and H, K-ATPase system could be altered under the physiological and patho-physiological states described as follows:

The high-affinity K site 122 accessible from the cell exterior or lumen is the target for a number of drugs. These include the benzimidizoles such as nolinium bromide and SCH 28080 that interacts competitively with the high-affinity K site thus effectively blocking the turnover of the proton pump.

There is also a low-affinity K site (trans-pNPPase site in FIG. 3) accessible from the cell exterior that is responsible for controlling the ion-channel function. Interference with this K site by compounds such as thiocyanate, isothiocyanate, oxycyanate and nitrite and their derivatives uncouple the system inhibiting the ion transport process. Of all the compounds affecting the K-site, preferably the organic thiocyanate and isothiocyanates are anti-mitotic.

The low-affinity ATP site 126 is accessible from the cell exterior acts as the regulator of the ion channel (FIG. 3) function. Homologues and analogues of ATP interfere with this low-affinity ATP site inhibiting the ion transport.

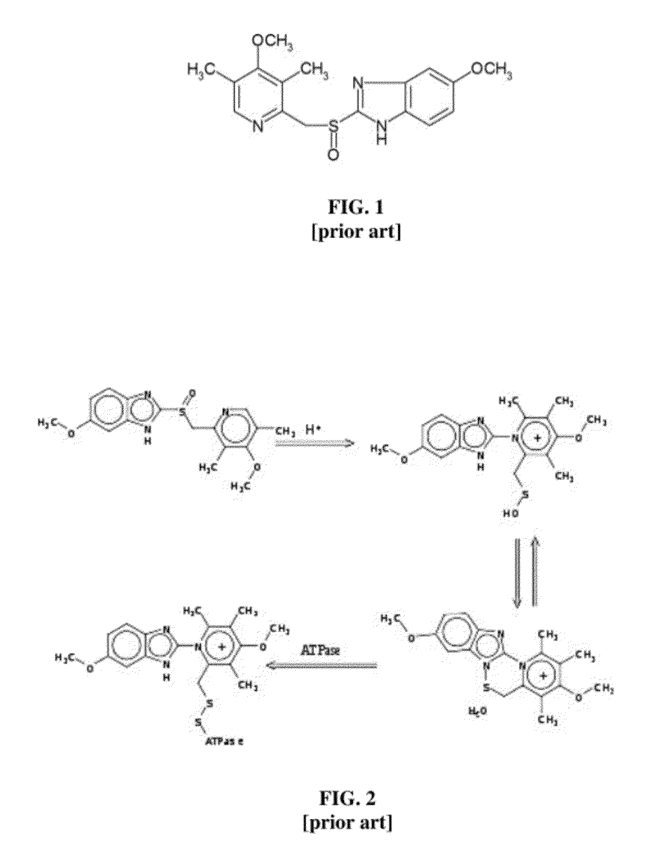

Binding of cardiac active steroids (CAS), such as ouabain, digoxin, bulfalin, digitoxin, not only inhibits the Na-pump by binding to the cell exterior but also at lower concentration (Xie, Z. and Askari, A. (2002),” Na, K-ATPAse as a signal transducer”, Euro. J. of Biochem. 269, 2434-2439, incorporated by reference herein) induces the formation of signaling modules through the activation of non-receptor (cytoplasmic) tyrosine kinases (Src). A CAS consists of a steroid nucleus and an unsaturated 5- or 6-membered lactone ring (FIG. 1). Cardioactive steroids are classified as a cardenolide (with a 5-membered lactone ring) or a bufadienolide (with a 6-membered lactone ring). Additionally, substitution of a sugar residue on the 3β-OH group at R1 creates a cardiac glycoside. The activation of Src in turn leads to several other cascades of reactions in cell specific manner, finally resulting in the expression of growth related genes.

The classical Post-Albers (PA) single-topology scheme was introduced in the sixties in an effort to explain the molecular mechanism of action of the Na, K-ATPase (Albers, 1967) and subsequently the gastric H, K-ATPase (Ray, T K and Forte, J G (1974), “Phosphorylated intermediates of the gastric H, K. ATPase reaction”, Biochim. Biophys. Acta 363, 320-339, incorporated by reference herein) system at the level of partial reactions (32P-intermediates), and has been instrumental for the phenomenal growth of both fields since then. However, many fundamental questions remain unanswered with the PA model and are discussed as follows: (a) No satisfactory explanation for the long standing observation (Taniguchi, K., Kaya, S., Abe, K. and Mardh, S. (2001); “Oligomeric nature of the Na, K transport ATPase”, J. of Biochem 129, 335-342, incorporated by reference herein) on half-of-the-site reactivity in the Na, K-ATPase reaction; (b) Presence of double ATP binding sites (Mignaco, J. A., Lupi, O. H., Santos, F. T., Barrabin, H and Scofano, H. M. (1996), “Two simultaneous binding sites for nucleotide analogues are kinetically distinguishable on the sarcoplasmic reticulum Ca-ATPase”, Biochemistry 35 (13), 3886-3891b, incorporated by reference herein) within the functional P2-ATPase complex cannot be explained by the PA model; (c) Simultaneous binding and transport of Na and K ions across the membrane (Antolovic, R., Hamer, E., Serpasu, E H, Kost, H., Linnertz, H. Kovarik, Z. and Schoner, W (1999); “Affinity labeling with Mg-ATP analogues reveals coexisting Na and K forms of the alpha subunit of the Na, K-ATPase”, FEBS Journal 261 (1), 181-189; Pauls, H., Serpersu, E. H., Kirch, U. and Shoener, W. (1986), “Chromium (III) ATP inactivating Na, K-ATPase supports Na—Na” and “Rb—Rb exchanges in everted red cells but not Na, K transport”, both incorporated by reference herein) cannot be explained by the PA model; and (d) The ouabain binding site at the ecto-domain involves extensive interaction with both α and β subunits (Hall, C. and Ruoho, A. (1980), “Ouabain binding site photoaffinity probes that label both subunits of Na, K-ATPase.”, PNAS 77, 4529-4533, incorporated by reference herein) of Na-pump that cannot be explained by the PA model.

The dual-topology of the Ray model 100 addresses all those concerns as above and thereby clarifies numerous published reports (“Dual Topology of the H, K-ATPase and Na, K-ATPase System: Testable model explaining data that remain baffling by conventional view”, likely to be published in the Biochemical Journal, 2011), that were previously unexplainable or hard to explain with the PA scheme. The dual-topology Ray model 100 is also consistent with numerous recent reports (Sebag, J. A. and Hinkle, P. M. (2009), “Region of melanocortin 2 (MC2) receptor-accessory protein for dual topology and MC2 receptor trafficking and signaling”, JBC. 284, 610-618; Lehner, D., Basting, B., Meyer, W., Haase, T., Manolikas, C., Kaiser, M., Karas, and C. Glaubitz (2008), “The key residue for substrate transport (Glu14) in the EmrE dimmer is asymmetric”, J. Biol. Chem. 283, 3281-3288; Sato, K., Pellegrino, M., Nakagawa, T., Nakagawa, T., Vosshall, L. B. and Touhara, K. (2008), “Insect olfactory receptors are heteromeric ligand gated ion channels”, Nature 452:1002-6; Padan, E. Kozachkov, L., Herz, K. and Rimon, A. (2009), “NhaA Crystal Structure: functional-structural insights”, J. of Experimental Biology 212, 1593-1603, all incorporated by reference herein) on the intrinsic state of various membrane proteins having similar dual orientation within the lipid bilayer, and has been suggested to arise from gene duplication (Lolkema, J. S., Dobrowolski, A. and Slotboom, D. J., “Evolution of antiparallel two domain membrane proteins: Tracing multiple gene duplication events in the DUF 606 family”, (2009) J. of Mol. Biol. 378, 596-606, incorporated by reference herein) making it a more general phenomenon.

Another notable feature of the new dual-topology model 100, as shown in FIG. 3, is that it provides for a bi-directional molecular motor (or pump) on the cell surface that uses the free energy of ATP hydrolysis for the uphill transport of H (or Na) and K ions across the membrane 102. Conformational stress within the adjoining α1 and α2 ion channels are, as shown in FIG. 3, caused by the rapid H (or Na)-dependent phosphorylation of the α1 subunit 110 (facing the cell interior) and resultant dephosphorylation upon binding of K to the α2-subunit 120 (facing the cell exterior) during each pump cycle. The rapidity of such phosphorylation and dephosphorylation processes during the ATPase turnover cause a harmonious shift of the adjacent ion channels lateral to the plane of the membrane causing a peristaltic effect for the simultaneous bidirectional transport of ions.

Dual-Topology Model for Drug Screening

The new dual-topology model 100, as shown in FIG. 3, deploys receptor function of the H, K-ATPase complex. Contrary to the single topology PA scheme, the Ray model 100, as shown in FIG. 3, offers ready access to the encountering effector molecules, such as the organic isothiocyanates, to bind to the specific ligand site 122 on the α2-subunit facing the lumen thereby exerting their effects. The effects could be either short lived (by direct uncoupling of the ion channel function at the trans K-pNPPase site) or long lived (gene regulation via the activation of protein kinase cascade) depending on the concentration (high or low) of the effector and time of exposure.

Additional studies on the binding of suspected target molecules to the purified H, K-ATPase, is a useful tool for the rapid screening of potential anti-ulcer and anti-cancer drugs, as shown in FIG. 4. A previous report (Nandi J and Ray T K (1986), “Mechanism of gastric antisecretory effects of thiocyanate: Further evidence for the thiocyanate induced impediment of gastric H, K-ATPase function”, Arch Biochem and Biophys, 244, 701-712, incorporated by reference herein; Ray, T. K., Bandyopadhyay, S., Ray, A. and Das, P. K. (1987), “K-pNPPase activity does not represent a partial step in the reaction sequence of the gastric H, K-ATPase system”, Ann. N.Y. Acad. of Sciences 494, 348-351, incorporated by reference herein) revealed specific binding of thiocyanate to the highly purified H, K-ATPase while inhibiting the K-pNPPase activity agrees well with this approach, but its value in drug screening is new, because the Ray model 100 provides a mode of action and receptor targets for new therapeutic compounds.

Anti-Ulcer and Anti-Cancer Properties

The aromatic thiocyanates and naturally occurring isothiocyanates include antitumor and/or anti-carcinogenic effects, which involve gene regulation thus enhancing the transcription of tumor suppressor proteins. Besides pumping H against a million-fold concentration-gradient the gastric H, the K-ATPase system also functions as receptors for signals receiving from the gastric pit and transmits them onto various intracellular compartments of the acid-secreting cells leading to gene activation. Such built-in feedback device is essential for the cells to function correctly in response to changing environment in the gastric pit. An aberration in this feedback system leads to diseases in due course.

Contrary to the inorganic salts of thiocyanate, the organic thiocyanate compound such as 2,4-dichlorophenyl thiocyanate (DCPT), possesses anti-proliferative property that initiates a signaling process that ultimately ends with the inhibition of mitosis by altering the microtubule morphology (Abraham, I, Dion, R L, Gottesman, M M and Hamel, E (1986) Proc. Natl. Acad. Sci. 83(18), 6839-6843, incorporated by reference herein). Such signaling effect of DPCT is exerted through its interaction with some genetically conserved domain on the cell-surface located α-subunit) P-2 ATPase system and is consistent with dual-topology model 100. In analogy with the known effect of ouabain as signal transducer (Xie, Z. and Askari, A. (2002), “Na, K-ATPAse as a signal transducer”, Euro. J. of Biochem. 269, 2434-2439, incorporated by reference herein) in the Na, K-ATPase system mentioned earlier, the binding of DPCT changes the interaction of the H, K-ATPase (α-subunit) with neighboring membrane proteins thru the activation of the Src (non-receptor tyrosine specific protein kinase) and thereby induce the activation of intracellular signaling cascades, causing the inhibition of cell division. Such antiproliferative effect has been included for a host of naturally occurring isothiocyanates, as described further below.

The thiocyanate or isothiocyanate moieties (also called pseudohalides due to similarities in chemical properties with the halogen family) are essential for both the anti-cancer and anti-ulcer activities. The modulating effect of pseudohalides on the P-2 ATPase system is analogous to that of another halide anion, fluoride, which activates the ubiquitous (plasma membrane bound) Adenylate Cyclase system (Downs, R W, Spiegel, A M, Singer, M, Reen, S and Aurbach, G D (1980), “Fluoride stimulation of adenylate cyclase is dependent on the guanidine nucleotide regulatory protein”, J Biol Chem 255 (3), 949-954, incorporated by reference herein) linked to the Protein Kinase A (PKA) mediated signaling module in all cells.

The antimitotic activity of thiocyanate has been demonstrated in tissue culture where the medication is in constant contact with the tumor cells. However, a similar medication in the stomach is washed away by the natural fluids and foods passing through the stomach and gastrointestinal system. Therefore, a delivery system with controlled release and retention in the stomach enhances the clinical benefit of the formulation.

The molecular mechanism of action of the Sodium thiocyanate (NaSCN, inorganic form) in gastric acid secretion. Thiocyanate is a potent inhibitor of gastric H transport in chambered bullfrog gastric mucosa, even though it does not inhibit the gastric H, K-ATPase mediated hydrolysis of ATP (as measured by the ATPase activity). The existing notion was that the SCN was acting as a protonophore and thus dissipating the proton gradient as HSCN. However, the Ray model determines that SCN does not inhibit the ATPase activity, but it does inhibit the associated K-pNPPase activity. The later could not be explained by the protonophore effect of SCN. Hence, a systematic investigation determined the precise mechanism of the SCN effect, (Nandi and Ray, 1986; Nandi, J and Ray, T. K. (1982), “Mechanism of action of gastric secretory inhibitors: Effects of SCN, OCN, NO₂ and NH₄ on H, K-ATPase mediated transport of H inside gastric microsomal vesicles”, Arch Biochem and Biophys, 216, 259-27, incorporated by reference herein).

The SCN moiety was binding to a specific site on the purified H, K-ATPase molecule and the bound SCN could be displaced by K demonstrating an antagonistic effect between these two. SCN appreciably inhibits the gastric K-pNPPase activity even though it does not inhibit the H, K-ATPase reaction. SCN does not interfere with the high-affinity K site responsible for the H, K-ATPase activity or turnover per se; however, SCN does interfere with a low-affinity K binding site responsible for the associated K-pNPPase reaction that appears to be responsible for the vectorial translocation of the generated protons, as shown in FIG. 3. SCN was uncoupling the proton pump.

In order to use SCN as a tool to control (or stop) acid secretion, the required concentration of co-cation (like Na) to be ingested (up to 1 gm) will be undesirable for human application. In this regard, the organic SCN, like DCPT (2,4-dichloro phenyl thiocyanate), is preferred over the inorganic ones as antisecretary agent; and additionally it has a clinical effect at much lower doses than the inorganic form. The demonstrated anti-proliferative (anti-mitotic) properties of the organic SCN makes them the drug of choice when coupled with the elimination of unnecessary (excess) inorganic cations, like Na, that is likely to be detrimental for human health.

The aromatic part (2,4-dichloro phenyl) of DCPT (organic form) includes a fairly lipid soluble molecule, hence bipolar, which makes DCPT and organic SCN more accessible to the actual binding site (Trans pNPPse site as shown in FIG. 3) at the membrane location. A lipid soluble SCN enables the molecule to be clinically more effective at lower concentrations than their inorganic (NaSCN) counterpart. In one embodiment, the clinical efficacy of the molecules is significantly enhanced by synthesizing more lipid soluble organic derivatives of the thiocyanates.

The aromatic SCN-derivatives like the DCPT is antimitotic (Bai, Ruo-Li, Chi Duanmu and Ernest Hamel. (1989), “Mechanism of action of the antimitotic drug: alkylation of sulfhydryl groups of beta-tubulin”, Biochim. Biophys. Acta 994, 12, incorporated by reference herein). As mentioned earlier the SCN moiety is essential for both the anti-ulcer and anti-cancer activity making these as the compounds of choice.

The Inhibition of Gastric Acid Secretion

The objective of this description is to demonstrate that the use of organic thiocyanate to inhibit the acid pump in the stomach is unobvious and therefore represents part of the invention described in this document. Aliphatic (alkyl and allyl) thiocyanates (of chain lengths greater than 6 carbon atoms) have obnoxious odors and are known for their pesticide properties (Murphy, D and Peet, J (1932) J of Econ and Entomology 25, 123; Bouquet, E., Salzberg, P and Dietz, H (1935) Ind. Eng. Chem. 27, 1342, incorporated by reference herein). Contrary to the aliphatic though, aromatic thiocyanates are not adverse. In fact, the aromatic thiocyanates and its various naturally occurring isomers, isothiocyanates, highly enriched in cruciferous vegetables have anti-cancer properties and are described further below.

The organic form of thiocyanate is more soluble in the membrane phase and therefore more bioavailable and clinically more effective than the inorganic form.

With the Post-Albers model, Sachs and collaborators (Jai, M S, Munson, K., Vagin, O. and Sachs, G. (2009) European J of Physiol. 457, pp 1432-2013) wrote that the enzyme pumps acid by a series of conformational changes from an E₁ (ion site in) to an E₂ (ion site out) configuration following binding of MgATP and phosphorylation; thus, following the single topology Post-Albers scheme. In the single-topology PA model, only one alpha subunit (designated as E) is considered to undergo rapid conformational changes generating E.ATP (ATP-bound state), E1 (high energy phosphate bound transition state) and E2 (another high energy phosphate-bound transition state) respectively. The E1 (with ion binding site facing inside the cell) has high affinity for H and low affinity for K, while the E2 (the same ion binding site now facing outside) has low affinity for H and high affinity for K respectively. As a result, H/K exchange under this PA model is proposed to occur at the cell exterior during each enzyme cycle in the following sequence: E+ATP E.ATP→E1−P→E2−P→E+P where E1 has strong affinity for H and E2 for K respectively causing H/K antiport in a cyclic fashion across the membrane.

Apell and coworkers (Witzke, A., Lindner, K., Munson, K. and Apell, H J (2010), “Inhibition of the gastric H, K-ATPase by Clotrimazole”, Biochemistry 49 (21), pp 4524-4532, incorporated by reference herein) wrote that the interaction of clotrimazole with H, K-ATPase introduces a single “dead end” branch added to the Post-Albers scheme in the E1 state of the pump, revealing their mental inclination for the PA model.” The antifungal, clotrimazole (CLT) is an imidazole compound having three separate benzene rings attached to a single carbon atom that makes it a hydrophobic molecule. The CLT has been demonstrated to be potent inhibitors of both Na—K-ATPase and H, K-ATPase systems by Apell's laboratory in their 2008 and 2010 articles respectively. It has been speculated that CLT acts on the K-binding site associated with the E2 conformation of the enzyme illustrated earlier.

Another prominent group dealing with the structure-activity relation of the gastric H, K-ATPase function (Abe, K., Tani, K., Nishizawa, T. and Fujiyoshi, Y. (2009, “Inter-subunit interaction of gastric H, K-ATPase prevents reverse reaction of the transport cycle”, EMBO Journal 28, pp 1637-1643, incorporated by reference herein) by the high resolution electron microscopy of 2-D crystal lattice wrote these results suggest that the beta-subunit N-terminus prevents the reverse reaction from E2P to EIP, which is likely to be relevant for the generation of a large H gradient in vivo situation, which following the classical Post-Albers scheme.

In view of the preceding quotations reflecting the mind-set of the scientists specializing in the study of the P-2 ATPase field, it is clear that the use of organic thiocyanates and isothiocyanates as gastric antisecretary agent that are consistent with Ray's dual-topology model (and disclosed in this US provisional Patent application) is highly unlikely to be obvious to others. Unlike the Ray dual-topology model, the PA scheme does not have a low-affinity ion-channel regulatory site facing the membrane exterior that interacts with thiocyanate for the observed uncoupling of ion transport

Gene Regulation and Anti-Inflammatory/Antitumor Properties

Some compounds of the present invention, for example, are known to possess potent anti-inflammatory or gene regulation (see below) capabilities. The anti-inflammatory activity down-regulates mitosis and thus supports an anti-cancer capability. Chronic inflammation up-regulates mitosis and thus is a precursor to tumor formation. To the extent that the compounds used in accordance with the present invention have anticancer activity, the effective blood level can be determined by analogy based on the effective blood level corresponding to anticancer activity.

The effective level can be chosen, for example, as that level (e.g., 10^{-11-10-7M}) effective to inhibit the proliferation of tumor cells in a screening assay. Similarly, the effective level can be determined, for example, on the basis of the blood or tissue level in a patient that corresponds to a concentration of a therapeutic agent that effectively inhibits the growth of human cancers in an assay that is clinically predictive of anticancer activity. Further, the effective level can be determined, for example, based on a concentration at which certain markers of cancer in a patient's blood are inhibited by a particular compound that inhibits cancer. Alternatively, the effective level can be determined, for example, based on a concentration effective to slow or stop the growth of a patient's cancer, cause a patient's cancer to regress or disappear, render a patient asymptomatic to a particular cancer, or improve a cancer patient's subjective sense of condition. The anticancer effective level can then be used to approximate (e.g., by extrapolation), or even to determine, the level which is required clinically to achieve a ATPase inhibiting-effective blood, tissue, and/or intracellular level to effect the desired medical treatment. It will be appreciated that the determination of the therapeutically-effective amount clinically required to effectively inhibit ATPase activity requires consideration of other variables that can influence the effective level, as discussed herein. When a fixed effective amount is used as a preferred endpoint for dosing, the actual dose and dosing schedule for drug administration can vary for each patient depending upon factors that include, for example, inter-individual differences in pharmacokinetics, drug disposition, metabolism, whether other drugs are used in combination, or other factors described herein that effect the effective level.

One skilled in the art can readily determine the appropriate dose, schedule, or method of administering a particular formulation, in order to achieve the desired effective level in an individual patient. One skilled in the art also can readily determine and use an appropriate indicator of the effective level of the compounds of the present invention. For example, the effective level can be determined by direct analysis (e.g., analytical chemistry) or by indirect analysis (e.g., with clinical chemistry indicators) of appropriate patient samples (e.g., blood and/or tissues). The effective level also can be determined, for example, by direct or indirect observations such as urine acidity, change in bone density, decrease in ocular pressure, or by the shrinkage or inhibition of growth of a tumor in a cancer patient (e.g., if the compound in question has anticancer activity). There are many references in the art that describe the protocols used in administering active compounds to a patient in need thereof. For example, the protocols used in the administration of anticancer agents to patients are described in “Cancer Chemotherapy: Principles and Practice” ed., Chabner and Collins, J. B. Lippincott, 1990, especially chapter 2, by J. B. Collins.

The isothiocyanates (pseudohalides), the glucosinolate precursor of sulforaphane (SFN) inhibit cell proliferation and induce apoptosis (programmed cell death) in a variety of cancer cell lines “Glucosinolates, structures and analysis in food”, Analytical Methods 2, 310-325; Hoist, B and Williamson, G. (2004), “A critical review of the bioavailability of glucosinolates and related compounds”, Natl. Prod. Rep. 21 (3), 425-447, incorporated by reference herein; Bonnesen, C, Eggleston, I M, and Hayes, J D (2002), “Diatary Indoles and Isothiocyanates that are generated from cruciferous vegetables can both stimulate apoptosis and confer protection against DNA damage in human colon cell lines”, Cancer Res. 61 (16), 6120-6130; Nho, C W, Jeffery, E. (2001), “Synergistic upregulation of phase II detoxification enzymes by glucosinolate breakdown products in cruciferous vegetables”, Toxicol. Appl. Pharmacol. 174(2), 146-152; Shapiro, T A, Fahey, J W, Wade, K L, Stephenson, K K and Talalay, P. (2001), “Chemoprotective glucosinmolates and isothiocyanates of broccoli sprouts: Metabolism and excretion in humans”, Cancer Epidemiolo. Biomarkers & Prevent. Vol. 10, 501-509, incorporated by reference herein). Isothiocyanates protect the cells against DNA damage by reactive oxygen species (ROS) and carcinogens (Zhang Y., Kensler T. W., Cho C-G., Posner G. H., Talalay P (1994), “Anticarcinogenic activities of sulforaphane and structurally related synthetic norbornyl Isothiocyanates”, Proc. Natl. Acad. Sci. USA, 91: 3147-3150; Fahey J. W., Zhang Y., Talalay P (1997), “Broccoli sprouts: an exceptionally rich source of inducers of enzymes that protect against chemical carcinogens”, Proc. Natl. Acad. Sci. USA, 94: 10367-1037, incorporated by reference herein). These effects of isothiocyanates are caused by the expression of critical enzymes such as UDP glucuronosyl tranaferase, quinine reductase and glutamate cystein ligase, known as the Phase II enzymes (Fahey et al, 1999).

The DNA of these phase II enzymes contains a specific DNA sequence called antioxidant response element (ARE); and the isothiocyanates enhance the transcription of the ARE. These isothiocyanates (also known as pseudohalides due to similarities with the halogens, F, Cl, Br and I) work in tandem with the H, K-ATPase molecule in the said downstream signaling scheme involving gene expression, as indicated below.

Both of the H, K-ATPase and Na, K-ATPase belong to the same P-2 ATPase family sharing 63% sequence homology with many conserved domains (Blanco, G and Mercer, R W (1998), “Isozymes of the Na, K-ATPase: Heterogeneity in structure, diversity in function”, 275, F633-650, incorporated by reference herein). Analogous to the cardiac glycoside (ouabain) that inhibits the ubiquitous plasmalemmal Na, K-ATPase system, the derivatives of thiocyanates and isothiocyanates (or the pseudohalides) also inhibit the gastric H, K-ATPase a member of the same P-2 ATPase-family by binding to specific (and conserved) extracellular (as shown in FIG. 3) site (Trans-pNPPase location) on the enzyme.

Also, similar to binding of ouabain (to the Na, K-ATPase), the binding of isothiocyanates to the H, K-ATPase system relays extracellular signals mediated by the activation of Src (kinase) inside the cells leading to the activation of the growth related proto oncogenes. The nature and state of the downstream-signaling pathways leading to gene activation depends on the nature of the concerned ligands, the nature of the specific site within the P-2 ATPase alpha subunit (conserved site), and the resultant communication with the neighboring nonreceptor tyrosine kinases (Src) located within the cytoplasm. Thus, the downstream signaling is different for different ligands, such as the cardiac glycosides or the isothiocyanates, due to differences in the nature of binding and hence the ending signal will be tissue specific. Therefore, the model 100, as shown in FIG. 3 provides a framework with which various anti-cancer and anti-ulcer drugs could be more efficiently and effectively screened for potential therapeutic or diagnostic uses for human or animal health.

Similarly the components of the formulation, green tea, curcumin, thiocyanate, isothiocyanate act to inhibit mitosis taken either individually but more clinically effective if taken together. The actives listed above provide clinical benefits using different physiochemical pathways. For example, curcumin inhibits lipoxygenase and cyclooxygenase 2, enzymes that are responsible for the synthesis of the pro-inflammatory leukotrienes, prostaglandins, and thromboxanes (Ammon H P, Safayhi H, Mack T, Sabieraj J., “Mechanism of antiinflammatory actions of curcumine and boswellic acids”. J Ethnopharmacol. 1993; 38(2-3):113-9. It also inhibits AP-1 mediated transcription related to cytokine regulation in vitro (Xu Y X, Pindolia K R, Janakiraman N, Chapman R A, Gautam S C., “Curcumin inhibits IL1 alpha and TNF-alpha induction of AP-1 and NF-kB DNA-binding activity in bone marrow stromal cells”. Hematopathol Mol. Hematol. 1997; 11(1):49-62) and suppresses inducible nitric oxide synthase (iNOS) in activated macrophages (Pan M H, Lin-Shiau S Y, Lin J K., “Comparative studies on the suppression of nitric oxide synthase by curcumin and its hydrogenated metabolites through down-regulation of IkappaB kinase and NFkappaB activation in macrophages”. Biochem Pharmacol. 2000; 60(11):1665-76.)), processes that promote inflammation. As lipid peroxides also promote inflammation, curcumin's antioxidant effects also serve to decrease inflammation.

In contrast green tea uses different physiochemical pathway than the other actives to provide its clinical benefits. Tristetraprolin (TTP/ZFP36) family proteins have anti-inflammatory activity by binding to and destabilizing pro-inflammatory mRNAs such as Tnf mRNA (“Green tea increases anti-inflammatory tristetraprolin and decreases pro-inflammatory tumor necrosis factor mRNA levels in rats” by Heping Cao, Meghan A Kelly, Frank Kari, Harry D Dawson, Joseph F Urban Jr, Sara Coves, Anne M Roussel and Richard A Anderson; Journal of Inflammation 2007, 4:1). Studies have shown that the most abundant catechin of green tea, (−)epigallocatechin-3-gallate (EGCG), strongly inhibits neutrophil elastase. Neutrophil cells in the blood play an essential role in host defense and inflammation. This is important since the latter can trigger and sustain the pathogenesis of a range of acute and chronic diseases. Also demonstrated that 1) micromolar EGCG represses reactive oxygen species activity and inhibits apoptosis of activated neutrophils, and 2) dramatically inhibits chemokine-induced neutrophil chemotaxis in vitro; 3) both oral EGCG and green tea extract block neutrophil-mediated angiogenesis in vivo in an inflammatory angiogenesis model, and 4) oral administration of green tea extract enhances resolution in a pulmonary inflammation model, significantly reducing consequent fibrosis. These results provide molecular and cellular insights into the claimed beneficial properties of green tea and indicate that EGCG is a potent anti-inflammatory compound with therapeutic potential (The Journal of Immunology, 2003, 170: 4335-4341. “Neutrophil Restraint by Green Tea: Inhibition of Inflammation, Associated Angiogenesis, and Pulmonary Fibrosis”, Massimo Dona, Isabella Dell'Aica, Fiorella Calabrese, Roberto Benelli, Monica Morini, Adriana Albini, and Spiridione Garbisa).

Screening of Potential Drugs

The Ray model 100 has led to a screening-method for human and veterinary therapeutic compounds and molecules. In one embodiment, FIG. 4 will save academic and commercial researcher's time and resources currently spent on potential drug candidates later found to be ineffective in preventing or treating conditions such as persistent chronic gastritis, ulcer, stomach cancer and/or other indications in human or veterinary health.

The Alpha2 (α2) subunit (as shown in FIG. 3) has the high-affinity K site (critical for H, K-ATPase turnover), one of the two low-affinity K-pNPPase sites (acting as H/K coupler), and the low affinity ATP site (pump regulator) —these sites are freely accessible from the lumen and are in turn open to the gastric pit. Thus, these prime targets for the H-pump function are available for binding with external ligands providing a convenient means for the screening of potential drugs described below and which is illustrated in the following flowchart, as shown in FIG. 4.

The screening method generally comprises Step 1 that includes testing the candidate ligands molecule or compound for anti-secretary activity, with and without K, using experiments such as the in vitro bullfrog gastric mucosa (Ray, T L and Tague, L L (1978), “Role of K-stimulated ATPase in H and K transport by Bull Frog Gastric Mucosa in vitro”, Proc. Symp. Gastric Ion Transport, Upsala 1977. Acta Physiol. Scand. Special Suppl, 1978, 283-292, incorporated by reference herein) system following its incorporation into the secretary solution; eliminating molecules that do not show appreciable antisecretary activity or antagonism to K; and if the tested ligands show both antisecretary activity and antagonism to K, then test the molecules with antisecretary activity found in Step 1b for their inhibition of the H, K-ATPase and K-pNPPase activities. The method further comprises Step 2 that includes testing to determine if a candidate molecule inhibits the H, K-ATPase activity (inhibits the pump turnover), then test in an appropriate animal model(s) for the antisecretary activity and select the best candidates for clinical trial, as in Step 6. Such compounds are not known to have anti-cancer effects as of the date of this disclosure (but might still be used as an anti-ulcer and antisecretary agent).

If a candidate molecule does not inhibit the H, K-ATPase but inhibits the K-pNPPase activity (acting as uncoupler of the pump), then continue with Step 3 and/or Step 4. These molecules can be expected to have anti-cancer and antisecretary (and/or anti-ulcer) effects.

Step 3 includes testing in an appropriate animal model(s) for the antisecretary activity and selecting the best candidates for clinical trial, Step 6.

Step 4 includes testing the antisecretary (and/or anti-ulcer) molecules selected in Step 3 in an appropriate cancer cell tissue-culture system (s) for any anti-proliferative activity and proceeding to Step 5 for molecules showing appreciable positive effects.

Step 5 includes testing the selected anti-cancer agents from Step 4 with validated animal tumor model(s) for pre-clinical oncology studies and performing the sets of standardized tests (involving for example, molecular pharmacology, tumor biology and histopathology) to assess their overall therapeutic value, and advancing the top candidate(s) to Step 6.

Step 6 includes conducting clinical trial(s) as anti-ulcer; antisecretary and/or anti-cancer; or other indications.

If an insufficient number of candidates survive any screening step, then go back a step or more and test lower-ranked candidates or run new candidates through the earlier steps of the screening method, as indicated in FIG. 4.

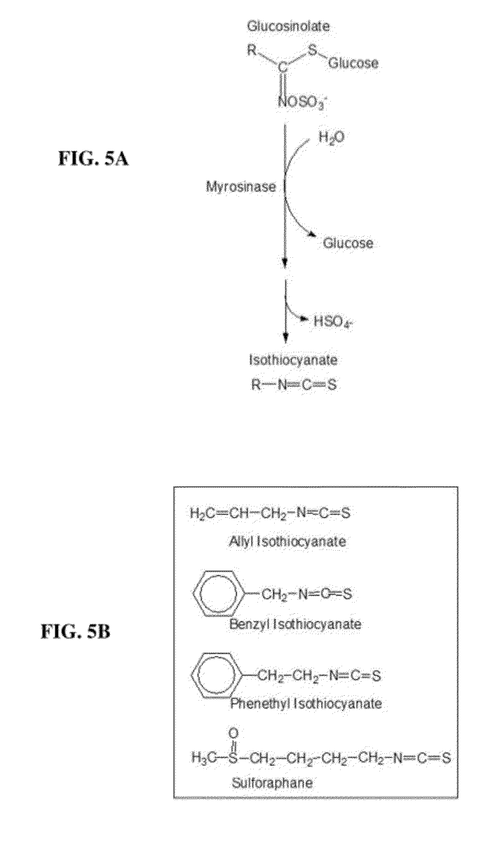

The isothiocyanates (—N═C═S) moiety includes the antisecretary and anti-cancer activities. The isothiocyanates are widely present as glucosinolates (Sulfur rich anionic metabolites) in cruciferous vegetables and are released by the endogenous plant enzyme, myrosinase (β-thioglucoside glucohydrolase) following damage or disintegration of the cells during food preparation, cooking or chewing. Myrosinase-catalyzed hydrolysis of the glucosinolates yield glucose and an unstable intermediate that spontaneously rearranges itself following the release of a molecule of H₂SO₄ with the formation of isothiocyanates as shown in FIG. 5a.

The glucosinolates are characterized by a core sulfated isothiocyanate group which is conjugated to thioglucose moiety having varying substitutions and a further R-group of diverse origin generating a host of different forms (Clark, 2010) found in nature. FIG. 5b shows the four primary types of isothiocyanates that are enriched in common cruciferous vegetables. There are also numerous synthetic thiocyanates and isothiocyanates that are available in the market (http://chemicalland21.com) for potential drug usage.

Compositions and Encapsulation Delivery

The ATPase inhibiting compounds of the present invention can be included in a composition, e.g., a pharmaceutical composition. The composition can be produced by combining one or more compounds of the present invention with an appropriate pharmaceutically-acceptable carrier, and can be formulated into a suitable preparation. Suitable preparations include, for example, preparations in solid, semi-solid, liquid, or gaseous forms such as tablets, capsules, powders, granules, ointments, solutions, suppositories, injections, inhalants, and aerosols, and other formulations known in the art for their respective routes of administration. In pharmaceutical dosage forms, a compound of the present invention can be used alone or in appropriate association, as well as in combination, with other pharmaceutically active compounds, including other ATPase inhibiting compounds, as described herein.

Any suitable carrier can be utilized. Suitable carriers include pharmaceutically or physiologically acceptable carriers. The following methods and carriers are merely exemplary and are in no way limiting. In the case of oral preparations, a compound of the present invention can be administered alone or in combination with a therapeutically effective amount of at least one other compound. Compositions used in accordance with the present invention can further include at least one additional compound other than a compound of the present invention, for example, an additional ATPase inhibitor (e.g., a concanamycin or a bafilomycin) or even an anticancer agent. The active ingredient(s) can be combined, if desired, with appropriate additives to make tablets, powders, granules, capsules, or the like.

Suitable additives can include, for example, conventional additives such as lactose, mannitol, corn starch or potato starch. Suitable additives also can include binders, for example, crystalline cellulose, cellulose derivatives, acacia, corn starch, or gelatins; disintegrants, for example, corn starch, potato starch or sodium carboxymethylcellulose; with lubricants such as talc or magnesium stearate. If desired, other additives such as, for example, diluents, buffering agents, moistening agents, preservatives, and/or flavoring agents, and the like, can be included in the composition.

The compounds used in accordance with the present invention can be formulated into a preparation for injection by dissolution, suspension, or emulsification in an aqueous or nonaqueous solvent, such as vegetable oil, synthetic aliphatic acid glycerides, esters of higher aliphatic acids, or propylene glycol (if desired, with conventional additives such as solubilizers, isotonic agents, suspending agents, emulsifying agents, stabilizers, and preservatives). The compounds of the present invention also can be made into an aerosol formulation to be administered via inhalation. Such aerosol formulations can be placed into pressurized acceptable propellants such as dichlorodifluoromethane, propane, nitrogen, and the like.

The compounds of the present invention can be formulated into suppositories by admixture with a variety of bases such as emulsifying bases or water-soluble bases. The suppository formulations can be administered rectally, and can include vehicles such as cocoa butter, carbowaxes, and polyethylene glycols, which melt at body temperature, but are solid at room temperature.

Unit dosage forms for oral or rectal administration such as syrups, elixirs, and suspensions can be provided wherein each dosage unit, e.g., teaspoonful, tablespoonful, tablet, or suppository contains a predetermined amount of the composition containing the compound of the present invention. Similarly, unit dosage forms for injection or intravenous administration can comprise a composition as a solution in sterile water, normal saline, or other pharmaceutically acceptably carrier.

The term “unit dosage form” as used herein refers to physically discrete units suitable as unitary dosages for human and animal subjects, each unit containing a predetermined quantity of at least one compound or compounds of the present invention (alone or, if desired, in combination with another therapeutic agent). The unit dosage can be determined by methods known to those of skill in the art, for example, by calculating the amount of active ingredient sufficient to produce the desired effect in association with a pharmaceutically acceptable carrier. The specifications for the unit dosage forms that can be used in accordance with the present invention depend on the particular effect to be achieved and the particular pharmacodynamics associated with the compound(s) in the individual host.

Pharmaceutically acceptable carriers, for example, vehicles, adjuvants, excipients, or diluents, are accessible to those of skill in the art and are typically available commercially. One skilled in the art can easily determine the appropriate method of administration for the exact formulation of the composition being used. Any necessary adjustments in dose can be readily made by a skilled practitioner to address the nature or severity of the condition being treated. Adjustments in dose also can be made on the basis of other factors such as, for example, the individual patient's overall physical health, sex, age, prior medical history, and the like.

In one embodiment, the method of the present invention includes co-administering a therapeutically effective amount of at least one compound of the present invention in combination with a therapeutically effective amount of at least one additional compound other than a compound of the present invention. For example, a compound of the present invention can be co-administered with an additional ATPase inhibitor (e.g., a concanamycin or a bafilomycin, as further described below), or with an anticancer agent (e.g., to inhibit the development of cancer cell resistance to the anticancer agent).

The compounds of the present invention can be administered by any suitable route including, for example, oral administration, intramuscular administration, subcutaneous, intravenous administration, or the like. For example, one or more ATPase inhibitors of the present invention (or a composition thereof) can be administered as a solution that is suitable for intravenous injection or infusion, a tablet, a capsule, or the like, or in any other suitable composition or formulation as described herein.

The ATPase “limiting-effective amount” is the dose necessary to achieve an ATPase “limiting-effective level” of the active compound in an individual patient. The ATPase inhibiting-effective amount can be defined, for example, as that amount required to be administered to an individual patient to achieve a ATPase inhibiting-effective blood level, tissue level, and/or intracellular level of a compound of the present invention to effect the desired medical treatment.

When the effective level is used as the preferred endpoint for dosing, the actual dose and schedule can vary depending, for example, upon interindividual differences in pharmacokinetics, drug distribution, metabolism, and the like. The effective level also can vary when one or more compounds of the present invention are used in combination with other therapeutic agents, for example, one or more additional ATPase inhibitors, anticancer compounds, or a combination thereof. Moreover, the effective level can vary depending upon the disease for which treatment is desired. For example, the effective level for the treatment of osteoporosis may vary relative to the effective level required for the treatment of abnormal urinary acidification, or for the inhibition of fertility.

An encapsulation system is described herein that keeps the medication in the stomach for extended periods of time to expose the tumor cells to the antimitotic treatment, in one embodiment of the invention.

One embodiment of the encapsulation system comprises an inner core, composed substantially of polysebacic acid (PSA), wherein the therapeutic agents are trapped or disposed inside the inner core. A particularly dense outer coating of polyethylene glycol (PEG) molecules is linked to the PSA inner core, which allows the therapeutic agent to diffuse through mucus nearly as easily as if it were moving through water and also permits the drug to remain in contact with affected tissues for an extended period of time.

Another embodiment of the encapsulation system for the release of the isothiocyanate and thiocyanates for anti-secretion, anti-inflamation and anti-mitotic activity comprises a plurality of nanoparticles or microparticles for a stomach-specific controlled release muco-adhesive drug delivery system prepared by ionotropic gelation of gellan beads with gellan gum. Aqueous solution of pectin and gellan gum (1.0-1.5% w/v) may be prepared with deionized water and was successively dispersed in to aqueous slurry of sodium alginate (2.0-2.5% w/v) with continues stirrer. The drug formulation (0.90% w/v) may be dispersed uniformly into 20 ml of the polymeric blended mixture with continuous stirring until a uniform dispersion is obtained. The mixture is then emulsified with 05-15 w/v of light mineral oil using Silverson emulsifier maintained continuous stirring with the oil at 500 rpm for 5 min. The resultant drug-loaded emulsions may be dropped through a syringe needle into 100 ml of 0.45 mol ml⁻¹ of calcium chloride (CaCl₂) solution, which may be kept under stirring to improve the mechanical strength and also to prevent aggregation. Immediate formation of small micro gel beads of the active formulation may be loaded sodium alginate blended, either with gellan gum or pectin gel beads after a particular curing time. The stomach-specific controlled release muco-adhesive drug delivery system has a percent loading (entrapment efficiency) in the range of 32% to 46% w/w in acidic medium, which increased up to 60% to 90% w/w in alkaline medium. A Chitosan coating forms a polyelectrolyte-complex film on the exterior of ionotropic gelation to increase the entrapment efficiency to produce a drug release over several hours. Alternative coatings may be applied by coating parameters including a 5-10% (w/v) ethyl cellulose (EC) solution in acetone and coating times is fixed. Gel beads may be placed in a fluidized bed dryer and the coating solution is sprayed on the fluidized beads using a spray gun for a period of time at an air inlet speed of ˜220 m/s at room temperature. The beads may be dried at room temperature for a period of ˜24 hr until all solvent was evaporated, leaving a film of EC coat on the gel bead.

Alternatively, the plurality of nanoparticles can be organic or polymer based and substantially spherical in shape and can have a diameter from about 0.1 nanometers (nm) to about 1000.0 nm. The nanoparticles are not, however, limited to being spherical in shape. Thus, the nanoparticles are asymmetrical in shape. If the nanoparticles are asymmetrical in shape, the largest cross-sectional dimension of the nanoparticles can be from about 0.1 nanometers (nm) to about 1000.0 nm in length. Nanoparticles can be solid, hollow or partially hollow and can be spherical or asymmetrical in shape. Optionally, the cross section of an asymmetric nanoparticle is oval or elliptical. As one skilled in the art will appreciate, however, other asymmetric shapes can be used. The nanoparticles can comprise shelled or multi-shelled nanoparticles. Shelled or multi-shelled nanoparticles can have targeting ligands conjugated to the shell material wherein the targeting ligand has an affinity for or binds to a target site in a subject or ex vivo. Such shelled or multi-shelled nanoparticles can be, made, for example, using techniques known in the art, for example, as described in Loo et al., “Nanoshell-Enabled Photonics-Based Imaging and Therapy of Cancer,” Tech. Cancer Res. and Treatment, (2004) 3(1) 33-40, which is incorporated herein by reference for the methods taught herein. Further, Oldenburg et al., “Nanoengineering of Optical Resonances,” Chemical Physics Letters (1998) 288, 243-247, is incorporated herein for methods of nanoshell synthesis. Shelled nanoparticles may be coupled with antibodies or other targeting agents to target a particular ATPase or parietal cell. Biodegradable polymers may include, but are not limited to, thermoplastic aliphatic poly (esters) like poly (lactide) (PLA) and poly (glicolide) (PGA) and copolymers of lactide and glycolide like poly (lactide-co-glycolide) (PLGA). PLA materials are less crystalline and less hydrophilic than copolymers (PLGA). Other biodegradable polymers are poly(glycerol-co-sebacate), mixing of poly(propylene carbonate) (PPC) with octadecanoic acid (OA) produces PPC-OA-x complexes. reverse theromogelling biodegradable polymers based on aliphatic polyesters, polyphosphazenes, poloxamer derivatives, polysaccharides, polypeptides, poly(propylene phosphate)s, polyorthoesters, polycarbonates, polycyanoacrylates, and poly(N-(2-hydroxyethyl)methacrylamide-lactate), N,N′-dimethylethylenediamine and 4-hydroxybenzyl alcohol linked by carbamate linkages, poly(propylene fumarate) and the crosslinking agent propylene fumarate-diacrylate, injectable nanocomposites made of biodegradable poly(propylene fumarate) and the crosslinking agent propylene fumarate-diacrylate, diblock copolymer of poly(sebacic acid) and poly(ethylene glycol) (PSA-PEG).

Another embodiment of the encapsulation system comprises beta Cyclodextrin to increase the solubility and transport of the thiocyanate through the cell membrane. Tissue culture studies have demonstrated significant increases in transport across the membrane of the therapeutic molecule. (Murali Mohan, Y., Jaggi, M., and Chauhan, S. (2010)’ “Beta-Cyclodextrin-curcumin self-assembly enhances curcumin delivery in prostate cancer cells”, Colloids and Surfaces B: Biointerfaces Vol. 79, 113-125, incorporated by reference herein). Alternatively, other cyclodextrins may be employed to encapsulate the active formulation, including, but not limited to α-cyclodextrin: six membered sugar ring molecule; β-cyclodextrin: seven sugar ring molecule; γ-cyclodextrin: eight sugar ring molecule, or any compound including 5 or more α-D-glucopyranoside units linked 1->4.

Another embodiment of the delivery system comprises a plurality of positively-charged gelatin microparticles or microspheres to coat the mucus of the stomach and the active ingredient encapsulated in the positively-charged gelatin microspheres. The positively-charged gelatin microspheres dissolve in the fluids of the stomach and are accelerated by the presence of acid. (Wang, J, Tauchi, Y, Deguchi, Y, Morimoto, K, Tabata, Y and Ikada, Y (2000), “Positively charged gelatine microspheres as gastric mucoadhesive drug delivery system for eradication of H. Pylori”, Drug Delivery vol. 7 (4), 237-243, incorporated by reference herein). The plurality of microparticles or microspheres can have a diameter between 0.1 and 100 μm in size

Poly(lactide-co-glycolide) (PLGA), chitosan (CHT) and PLGA/CHT microparticles (MPs), PLA, PGA MPs may be prepared by emulsion, evaporation, precipitation or spray-drying techniques. The polymer (D, L-PLA, 100-200 mg) and the active drug (10-30 mg) were dissolved in a water-immiscible organic solvent (dichloromethane), which was emulsified in a PVA aqueous solution (1-5%) using an Ultraturrax T25 (13.500 rpm). Stirring was maintained at with stirrer/heat plate until organic solvent evaporated in atmospheric temperature (25±1 μC). Then, the microparticles may be obtained/separated by centrifugation and re-suspended in distilled water three times and freeze-dried until use.

Hydrogels are three-dimensional cross-linked networks that become soft and gel-like which have similar texture as some living tissues with good biocompatibility. Biodegradable hydrogels as drug carriers have the advantage of releasing entrapped drugs via both diffusion and matrix biodegradation mechanisms. Hybrid hydrogels could provide easily controlled hydrophilicity to hydrophobicity for the active formulation and acidic environments. Hybrid co-polymers could be achieved by AB or ABA block co-polymerization, grafting and semi-interpenetrating or co-polymerization between hydrophilic and hydrophobic precursors. For example, hydrophilic-hydrophobic, block and graft co-polymeric hydrogels (based on vinyl alcohol and hydroxy-ethyl methacrylate moieties) may be employed. A family of biodegradable hybrid hydrogels may consist of hydrophobic poly(D,L-lactic acid) diacrylate macromer (PDLLAM) and hydrophilic dextran derivatives of ally isocyanate (Dex-AI) for controlled release of biologically active agents. Or hydrophobic-hydrophilic hybrid hydrogels may consist of poly(ε-caprolactone) maleic acid and poly(ethylene glycol) diacrylate. Finally, photocross-linked hydrophobic-hydrophilic biodegradable hybrid hydrogels may be formulated from hydrophobic multiarm poly(ε-caprolactone) maleic acid (PGCL-Ma) and hydrophilic polysaccharide dextran derivatives of maleic acid (Dex-Ma) precursors.

Another embodiment of delivering the active treatments comprises a button pill operable to adhesively attach to the side of a tooth between the tooth and cheek. Preferably, the button pill is operable to attach to the lower jaw because saliva pools around the lower jaw. The button pill includes the active treatments that dissolve over time in the saliva and be swallowed and delivered to the stomach and intestines.

In one embodiment, the dosage of isothiocyanate or thiocyanate is an ATPase “inhibiting-effective amount”. In another embodiment, the dosage of isothiocyanate or thiocyanate is between about 100 to 500 mg, and for the divalent ions zinc, barium or calcium is between about 0.5 to 5.0 mg, respectively.

The oral composition can include inert diluents, adjuvants such as wetting agents, emulsifying and suspending agents, sweetening, flavoring and perfuming agents and also agents that provide controlled release of the active ingredients over time and at acid and more neutral pH's as in the gastrointestinal tract. The encapsulating system can be designed to dissolve and release the active ingredients in the low pH of the stomach or in the neutral pH of the small and large intestine. Variously, both encapsulating systems can be incorporated into one pill so active treatment is delivered to both the stomach and intestines. The formulation can also include a neutralizing component as calcium carbonate to neutralize secreted acid existing in the stomach and to prevent the secretion of additional acid with the other components.

The formulation provides a combination of several actives contained in one unit of a delivery system that can be delivered to the target tissue. This combination approach includes a delivery system with particular characteristic detailed below. This combination of actives can be simultaneously released at the extracellular surface of the cell membrane or in the cell to act in concert to achieve an improved clinical result. The actives can be a combination or all of the following, but not limited to: green tea, curcumin, thiocyanate, isothiocyanate, zinc and their equivalents. Each of these agents has an anti-inflammatory effect and each achieves this result through a different physiochemical pathway. Zinc has a potentiating effect on TC and ITC to reduce inflammation. Therefore all actives acting on a single cell simultaneously will have a heightened effect over a single agent to reduce inflammation. Reduced cellular inflammation also reduces the mitotic activity of the cell and supports an anti-cancer benefit.

Layered Tablet Formulation

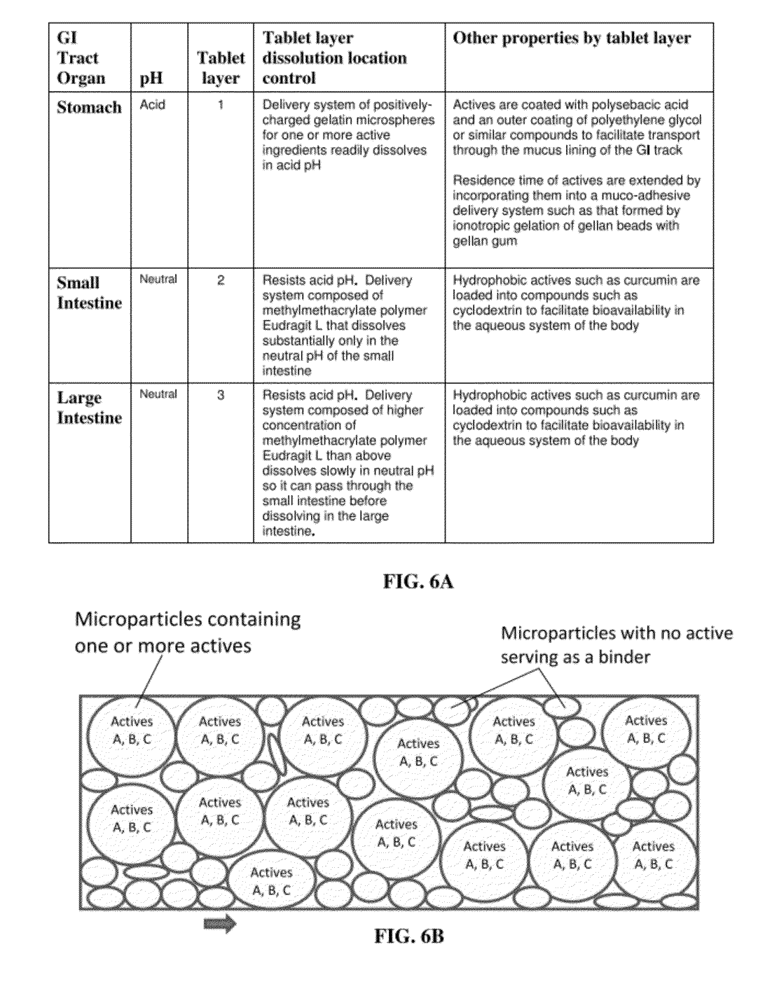

A tablet can be molded using high-speed production equipment resulting in one, two or three layers where each layer is a different powder. Referring to FIG. 6A each layer of the tablet dissolves in the stomach or intestine releasing microparticles filled with one or more actives. These microparticles are composed of a formulation that has a delayed release to allow it to travel through the blood to cells and then release the actives. A method of treating excess stomach acid, inflammation or cancer comprising a combination of active ingredients contained in an oral delivery system wherein the actives are contained in a tablet that is comprised of one, two or three separate layers, and each layer has a different delivery system and one or more chemically compatible actives may be in each layer. The tablet could be composed of one layer however the preferred embodiment is two or three layers. The delivery systems may include clinically in-active carrier material or materials that inhibit or facilitate dissolution and absorption and/or topical use of the layer in the stomach and others which facilitate dissolution and absorption and/or or topical use in the small or large intestine.

In one embodiment, the actives are coated with polysebacic acid and an outer coating of polyethylene glycol or similar compounds to facilitate transport through the mucus lining of the gastro-intestinal (GI) track. In another embodiment, the residence time of isothiocyanate, thiocyanates and other actives are extended by incorporating them into a muco-adhesive delivery system such as that formed by ionotropic gelation of gellan beads with gellan gum. In one embodiment, the hydrophobic actives such as curcumin are loaded into compounds such as cyclodextrin to facilitate bioavailability in the aqueous system of the body. Alternatively, the clinically significant concentration of the actives is extended by releasing them at two or more stages of the GI tract by the design of the delivery system. One or more outer layers of the tablet are made up of a delivery system of positively-charged gelatin microspheres for one or more active ingredients that substantially dissolves in the acid of the stomach for topical treatment and/or absorption into the bloodstream. The first or second layer of the tablet contains a delivery system composed of methylmethacrylate polymer Eudragit L that dissolves substantially only in the neutral pH of the small intestine, thereby releasing one or more of the active ingredients for topical treatment and/or absorption into the blood stream. The first, second or third layer of the tablet can contain more of the delivery system's carrier material (such as methylmethacrylate polymer Eudragit L) to delay the dissolution until this layer reaches the large intestine to release one or more actives for topical treatment and absorption into the blood stream or it can use a different carrier material that takes longer to dissolve in a neutral pH environment. The tablets may be a food supplement or a medical food. The tablets may be a pharmaceutical drug. Tablets may be one or more layers and may include any combination of layers described above. In one embodiment, each layer is a different formulation and there may be multiple actives in one layer if they don't react to each other. Some actives are better suited to high or neutral pH absorption. The delivery system achieves a higher absorption rate, longer times of clinically significant levels of the actives in the bloodstream or topical contact, and extended shelf-life. From FIG. 6a, the qualitative composition of layer 3 can and most likely is the same as layer 2. However, in layer 3, the release of the actives is delayed due to more carrier being present in layer 3.

The advantage of the three layers is that it provides release of actives for both topical treatment and absorption into the bloodstream through the stomach and the small and large intestines as the tablet contents move through the GI system. The topical treatment is clinically advantageous because it provides a high concentration of actives for anti-secretory and anti-inflammatory benefits. The absorption of the actives into the circulatory system supplements the topical benefit plus provides the same benefit systemically. The period of clinically effective concentration of the actives in the bloodstream is extended by the sequenced release in the stomach, small intestine and large intestine.

The three layer tablet delivery system includes each layer made from a different fine powder that is tamped in a mold in sequence and final high-pressure tamp to fuse the three layers together into one tablet. Its use may be to extend the shelf life of drugs and food supplement by keeping separate actives that chemically interact and thus reducing their effectiveness over time and shortening their shelf-life. Use of the layered tablet manufacturing capability to target topical delivery in the GI tract and to target the point of absorption for extending the absorption period to thereby extend the period of clinical benefit.

Referring to FIGS. 6A and 6B, one layer can be composed of a delivery system that dissolves in the acid of the stomach to release the actives in the stomach. A second layer can be composed of a delivery system that dissolves only in the neutral pH of the small intestine to release its actives. A third layer can be composed of a delivery system that dissolves only in neutral pH after 10 hours where the actives can be released in the large intestine. One tablet can thus be programmed to release the same or several different actives variously in the stomach, small intestine and/or large intestine. Examples of delivery systems to accomplish the release of one or more actives in different parts of the gastrointestinal tract are described below.

Formulation: Release in Neutral pH

Microparticles prepared using the following process showed release in neutral pH and limited release in acid (<10% release). This formulation plus actives would not dissolve in the stomach but would dissolve in neutral pH of the intestine. Changing the ratio of the components of the delivery system can delay the dissolution so the active is released in the large intestine. The microparticles were prepared using the pH-responsive methylmethacrylate polymer Eudragit L by an emulsion-solvent evaporation process. In the process active and polymer are dissolved in ethanol, and dispersed in a liquid paraffin external phase using sorbitan sesquioleate as stabiliser. The influence of the physicochemical properties of the molecules (solubility in the inner phase, partition coefficient [ethanol/paraffin]) on the distribution, encapsulation efficiency and pH-responsive dissolution behavior of the microparticles were examined. The drug that tended to partition in ethanol rather than liquid paraffin (riboflavin) was efficiently encapsulated and evenly distributed. In contrast, compounds which partitioned in favor of the liquid paraffin localized towards the surface of the microparticles and exhibited lower encapsulation efficiency (Suchada Nilkumhang^{a}, Mohamed A. Alhnan^{a}, Emma L. McConnell^{a} and Abdul W. Basit.: Drug distribution in enteric microparticles”, International Journal of Pharmaceutics Volume 379, Issue 1, 8 Sep. 2009, Pages 1-8.

Formulation 2: Releases in Acid Ph:

A delivery system that releases the actives in the acidic pH of the stomach is made by free radical polymerization of Poly[N-vinyl pyrrolidone-acrylic acid]-polyethylene glycol (Poly[NVP-AA]-PEG). Azobisisobutyronitrile (AIBN) was used for initiating the polymerization and N,N″-methylene bisacrylamide was employed as a crosslinking agent. The concentration of initiator and crosslinking agent can be varied with the reaction parameters to regulate the rate of release of actives (P Ravichandran^{a}, K. L Shantha^{a} and K. Panduranga Rao. “Preparation, swelling characteristics and evaluation of hydrogels for stomach specific drug delivery”. International Journal of Pharmaceutics. Volume 154, Issue 1, 12 Aug. 1997, Pages 89-94).

All microparticles can be made with Formulation 1 or 2. Microparticles without actives will have a formulation allowing rapid dissolution once at the target location, e.g. stomach or intestine. Microparticles with actives will have the formula adjusted to delay the release of the actives until the microparticle reaches the cells. In the case where the actives are to be delivered to the cells in the stomach some or all of the actives can be encapsulated with Formulation 1 since cells are neutral pH.

The characteristics of the preferred delivery system for this unique combination of therapeutic agents would be composed of a high molecular-weight carrier with a high degree of three-dimensional structures to entrap the various active molecules. Examples of delivery system that fit these requirements include the following, but not limited to: Large numbers of attached rings to form plates that are clustered together; multifunctional macromers based on poly (ethylene glycol) and poly (vinyl alcohol) polymerized to form degradable hydrogel networks; A hydrogel is a network of polymer chains that are hydrophilic and can be used as a drug delivery system since it changes its chemical properties with temperature, pH, ionic strength, ultrasonic sound, electric current and other forms of energy. One hydrogel, poly(N-isopropyl acrylamide)(pN1PAAm) and its various derivatives is temperature sensitive and thus serves as the stimulus to release the active. A zerogel is a solid formed from a gel by drying with unhindered shrinkage and high porosity and thus have a large surface area. The pore size (1-10 nm) can be varied to regulate the rate of release.

Polyacrylic acid with a high molecular weight in the millions or higher has the special characteristic of sticking to the mucosal lining of the gastrointestinal tract. The mixture of polyacrylic acid sodium salt (PAA) and Chitosan hydrochloride (HCS) creates a delivery system with muco-adhesive properties to release the active ingredient over protracted periods. The ionic interaction between PAA and HCS forms a substantive material. The rate of release and percent loading can be affected by the ration of the two ingredients for example a desirable ration is 1.3 to 1.0. They also can be affected by the molecular weight of the PAA from millions to billions of kDa. The higher the molecular weight the slower the release. The shape numerous side chains and the high number of carboxyl groups provide ionic and physical retention of actives. The free-radical polymerization of acrylic acid in the presence of Chitosan produces porous microspheres that can release actives at different rates depending on the pH. Polyacrylic acid can be used by itself for a delivery system however the characteristics of the system can be regulated more affectively when using one or more other components in the formulation. Cyclodextrin has the important feature of having a hydrophilic outer-surface to facilitate transport through the fluid system of the body to the target cell and an inner structure to hold the actives captive. Cyclodextrins are cyclic oligosaccharides that have the ability to form non-covalent complexes with a number of drugs and in so doing alter their physicochemical properties. In addition, the primary and secondary hydroxyl groups of the native (α, β, γ-) cyclodextrins are potential sites for chemical modification. Cyclodextrins are made up of a six, seven or eight sugar ring molecule with a hydrophilic exterior to facilitate mobility through the fluids of the body and cell membranes even for more hydrophobic actives that are loaded in the center. Starch microspheres also show a muco-adhesive quality that allows the active to be release over 10 to 15 times longer than if the delivery system was not utilized. The unique properties of dendrimers, such as their high degree of branching, multivalency, globular architecture and well-defined molecular weight, make them a good candidate drug delivery system for the combination of several actives listed above.

Dendrimers are repeatedly branched, roughly spherical large molecules. The types of dendrimers used are preferably a unimolecular micelle by encapsulating a pharmaceutical through the formation of a dendrimer-drug supramolecular assembly; including but not limited to the following: Poly (amidoamine) dendrimers (PAMAM) are synthesized by the divergent method starting from ammonia or ethylenediamine initiator core reagents; Radially layered poly(amidoamine-organosilicon) dendrimers (PAMAMOS) are inverted unimolecular micelles that consist of hydrophilic, nucleophilic polyamidoamine (PAMAM) interiors and hydrophobic organosilicon (OS) exteriors; PPI-dendrimers are “Poly (Propylene Imine)” describing the propylamine spacer moieties and are generally poly-alkyl amines, having primary amines as end groups, the dendrimer interior consists of numerous of tertiary tris-propylene amines; Tecto Dendrimer are composed of a core dendrimer, surrounded by dendrimers of several steps (each type design) to perform a function necessary for a smart therapeutic nanodevice; Multilingual Dendrimers include a surface that contains multiple copies of a particular functional group; Micellar Dendrimers are unimolecular micelles of water soluble hyper branched polyphenylenes. Alternatively, the formulation may be covalently attached to the periphery of the dendrimer to form dendrimer prodrugs, second the formulation may be coordinated to the outer functional groups via ionic interactions, or the dendrimers may be coating with alternative compounds for targeting to the ATPase.

Additional ATPase Inhibitors

The method of the present invention can be made more effective by administering other compounds, such as, for example, another ATPase inhibitor (e.g., a concanamycin and/or a bafilomycin, and others as described below), along with a compound of the present invention. The compounds of the present invention also can be co-administered with an anticancer agent, in which case the effective level desirably is the level needed to inhibit the ability of the cancer to develop resistance to the anticancer agent. Suitable anticancer compounds include, for example, all of the known anticancer compounds approved for marketing in the United States, and those that will become approved in the future, for which drug resistance thereto can be controlled by the inhibition of ATPase.

The unique ATPase limiting activity of the compounds of the present invention can be determined using any suitable method known in the art, for example, assay methods. A suitable assay method for measuring ATPase inhibitory activity is described, for example, in Chan et al., Anal. Biochem., 157, 375-380 (1986).

Alternatively, the unique ATPase-limiting activity of the compounds of the present invention can be demonstrated using the U.S. National Cancer Institute's (NCI)'s 60 cell-line, human tumor, disease-oriented screen, which can accurately predict the anticancer activity of chemical compounds. Significantly, the NCI 60 cell-line screen also is a powerful tool that can be used to predict other types of biological activity, not limited to anticancer activity.

The prediction of biological activity is based on the correlation of activity patterns generated in the NCI screen by compounds having known activity. The compounds compared in the correlation need not have particularly potent anticancer activity in order to display an activity pattern suitable for correlation in the NCI screen. Interestingly, compounds need not be structurally similar to one another in order correlate with each other in the NCI screen. Even if two structurally dissimilar compounds correlate strongly with each other in the NCI screen, they can be accurately predicted to have the same biological activity as each other in virtually any application, including non-cancer applications.

There are three classes of agents that effect the proton pump 1) K-antagonists (both low and high affinity), 2) Agents interfering with the ATP binding sites and 3) Inhibition by heavy metal and transitional metal ions. The heavy metal ions listed below have a synergistic effect with the other agents listed in items 1 and 2 and, thus, has an enhanced therapeutic effect to reduce acid secretion.

K-Antagonists generally comprise low-affinity K and high-affinity K antagonists. Low-affinity K antagonists include SCN, OCN and NO₂ (Nandi and Ray, 1882 and 1986) and the polyamines, Spermine and Spermidine (Ray et al, 1982). High-affinity K antagonists include Quinolizium compounds like nolinium bromide, (2-[3,4]-dichlorophenylamino quinolizium bromide (Ray et al, 1983), and SCH 28080 (Wallmark, B, Briving, C, Fryklund, J, Munson, K, Jackson, R, Mendlein, J, Rabon, E and Sachs, G (1987) Inhibition of gastric H, K-ATPase and acid secretion by SCH 28080, a substituted pyridyl (1, 2a)imidazole. J Biol Chem 262, 2077-2084, incorporated by reference herein) and derivatives

ATP-site Blockers generally comprise low-affinity and high-affinity ATP antagonists. Low Affinity ATP Antagonists: Adenosine-5-gamma-thiophosphate, AMP-PNP, Azido ATP, TNP-ADP, TNP-8-azido-ADP, Dansyl azido-ADP, (NH4)4-Cobalt-ATP, Azido-SL-ATP (relevant references are provided in the attached manuscript by Ray and Nandi), and CTP and ITP (unpublished data by Ray and coworkers). High-Affinity ATP Antagonists include FITC, Fluorscein Isothiocyanate (Farley et al. The amino acid sequence of a fluorescein-labeled peptide from the active site of (Na,K)-ATPase. (1984) J. Biol. Chem. 259, 9532-9535, incorporated by reference herein)

Heavy-metal ions generally comprise Ba, Ca, and Zn and show synergistic inhibition when used in combination with thiocyanate and isothiocyanate. Zinc (Zn) inhibits acid secretion by a mechanism quite different than Barium and Calcium. Zn at a concentration of 60 reduces the acid secretion to near zero (within 15 min) when added to the secretary solution of a chambered bull frog gastric mucosa, and the secretion can be promptly restored by 1 mM beta-mercapto-ehanol (βME), a thiol agent. Consistent with this observation the activity of the isolated H, K-ATPase and pNPPase were strongly inhibited by Zn; the former activity being ten times more sensitive than the latter. Zn at 2 μM abolished the H, K-ATPase activity while binding to about 16 n Moles of sulfhydryl (—SH) group per mg of membrane protein at the same time. The data suggest that Zn inhibits the proton pump (H, K-ATPase activity) by binding to some critical —SH group exposed to the lumen of the stomach.

Barium (Ba) shows slight stimulation of acid secretion at less than 1 mM concentration where as it blocks the acid secretion at higher concentration. Higher concentration of K competitively reverses the Ba inhibition showing a Ba/K antagonism (Ray, 1980; Ray, T K and Tague, L L (1980) Secretagogue induced transport of H and K by in vitro amphibian gastric mucosa. Biochem. Pharmacol. 29, 2755-2758, incorporated by reference herein). The gastric microsomal K-pNPPase activity also showed a parallel Ba/K antagonism. To the contrary, even though Calcium (Ca) shows strong inhibition of the K-pNPPase at concentrations comparable to Ba the effects were rather nonresponsive to higher K. These diverse mechanisms of action of the divalent cations, discussed as above, shows a synergistic inhibition in gastric acid secretion when used in combination.

Both thiocyanate (SCN) and Zinc (Zn) act as ATPase inhibitors or potent inhibitors of gastric-acid secretion when tested in chambered bull frog gastric-mucosa in vitro. However, SCN and ZN include different modes of operation. Due to such radical differences in their modus operandi (see below) on the gastric proton pump, SCN and Zn will show synergistic inhibition under therapeutic situation if used together.

While the thiocyanate (SCN) inhibition of gastric acid secretion can be reversed by raising the concentration of K-salt in the bathing medium, the latter was totally ineffective in the case of Zinc inhibition. Contrary to the thiocyanates however, the Zn inhibition could be reversed by the addition of beta-mercaptoehanol (βME), a thiol agent, into the secretary solution suggesting the involvement of some critical sulfhydryl (—SH) groups in gastric H transport. Quantitation of the sulfhydryl (—SH) groups within the gastric H, K-ATPase system before and during Zn inhibition revealed about 3 critical —SH groups per mole of the enzyme to be involved in the process.

Studies with the isolated gastric microsomal vesicles enriched in H, K-ATPase activity revealed detailed molecular insights into the modus operandi of the two types of inhibitors on the proton pump. Zinc abolishes the gastric H, K-ATPase activity at low (2 μM) concentration and also the associated K-pNPPase at ten-fold higher (20 μM) concentration signifying its primary action is on pump turnover, whereas thiocyanate (10 mM) was totally ineffective on the H, K-ATPase but abolished the K-pNPPase activity. Parallel to its effect on mucosal acid secretion mentioned earlier, the thiocyanate inhibition of the K-pNPPase activity was reversed by high K showing a competition between K and SCN. Further studies revealed that the pNPPase activity reflects the state of the transmembrane ion-channel within the proton pump (H, K-ATPase system), and inhibition of the pNPPase reaction by SCN is due to an uncoupling of the underlying H/K exchange mechanism.

Permanent Proton Pump Inhibitors

The method of the present invention can be made more effective by combing the actives with a permanent proton pump inhibitor. The permanent proton pump inhibitors stop the secretion of acid into the stomach for the life of the acid producing cell. Acid is secreted to digest food only after new cells are produced and that process can take over two weeks. During this time period, there can be inadequate levels of acid for normal digestion and thus absorption of nutrients, which can lead to the common side effects, associated these types of gastric-acid release inhibitors. In contrast, the plurality of actives listed above stops the secretion of acid only while the formulation of plurality of actives is present and can be cleared in about twelve to twenty four hours, which will allow the subsequent secretion of normal levels of stomach acid for digestion.

By combining these two types of proton pump inhibitors, a more normal level of acid can be obtained over an extended period of time for improved digestion and stomach comfort and also fewer side effects. Low doses of each type of the actives listed above and the permanent proton pump inhibitors will allow only some of the acid secreting cells lining the stomach to be permanently turned off and the balance of the cells will be temporarily inhibited until the high acid condition that is causing the discomfort is neutralized.

The chemical structure, chemical name and brand names are listed below for the permanent proton inhibitors. Each chemical has multiple brand names.

Omeprazole (brand names: Losec®, Prilosec®, Zegerid®, ocid, Lomac®, Omepral®, Omez®), indicated by the following chemical structure:

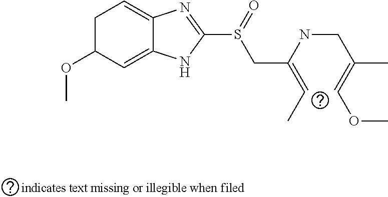

Lansoprazole (brand names: Prevacid®, Zoton®, Inhibitol®, Levant®, Lupizole®), indicated by the following chemical structure:

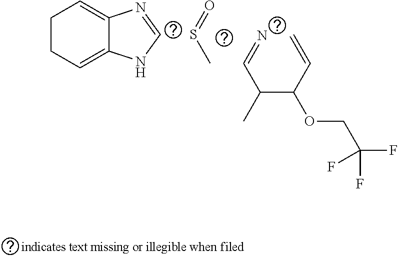

Dexlansoprazole (brand name: Kapidex®, Dexilant®) is chemically an enantiomer of lansoprazole, indicated by the following chemical structure:

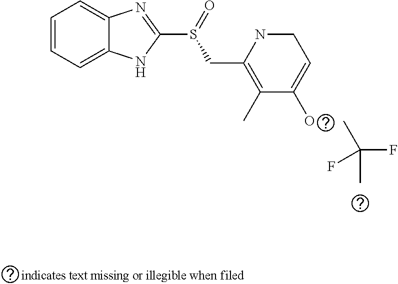

Esomeprazole (brand names: Nexium®, Esotrex®), indicated by the following chemical structure:

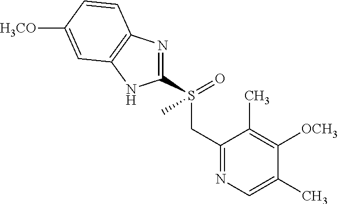

Pantoprazole (brand names: Protonix®, Somac®, Pantoloc®, Pantozol®, Zurcal®, Pan), indicated by the following chemical structure:

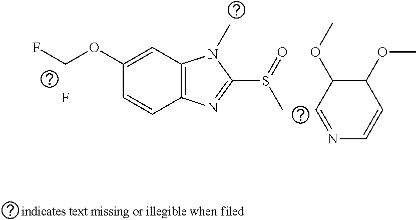

Rabeprazole (brand names: Zechin, Rabecid, Aciphex, Pariet, Rabeloc), indicated by the following chemical structure:

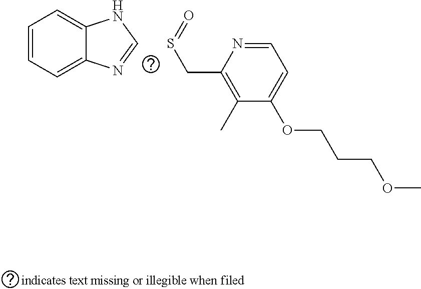

PPI molecules named above share the same Benzimidazole nucleus shown below.

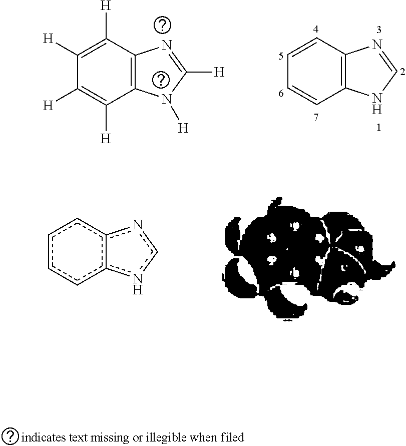

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A method of treating a subject that has an elevated level of stomach-acid secretion, inflammation, or cancer anywhere in the body by administering a composition to said subject where it is composed of one or more of a thiocyanate that has the structure of Formula (I) 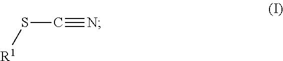
or an isothiocyanate that has the structure of Formula (II): 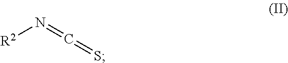
wherein R1 and R2 is an organic and lipid-soluble molecule.

2. The method of claim 1, wherein said isothiocyanate or thiocyanate is one or more of sulforaphane, sulfoaphene, erysolin, erucin, iberin, alyssin, berteroin, iberverin, or cheirolin.

3. The method of claim 2, wherein said composition is a food supplement, a dietary supplement, food additive or medical food.

4. The method of claim 1, wherein said composition is a pharmaceutical composition.

5. The method of claim 1, wherein the composition further comprises gastric acid release inhibitors selected from the group consisting essentially of: a benzimidazole nucleus that is common to gastric-acid release inhibitors on the market such as omeprazole, lansoprazole, dexlansoprazole, esomeprazole, pantoprazole and rabeprazole.

6. The method of claim 1, wherein the composition further comprises divalent cations selected from the group consisting essentially of: zinc, barium or calcium to provide a synergistic effect to enhance therapeutic effectiveness.

7. The method of claim 1, wherein said composition is encapsulated with a delivery system designed to provide prolonged release in the stomach or intestine.

8. The method of claim 1, wherein said composition is encapsulated with a delivery system that contains nanoparticles or microparticles to penetrate the mucus lining or a system that anchors to the mucus to provide prolonged release of active ingredients over several hours.

9. The method of claim 1, wherein the composition down regulates the Na—K ATPase system.

10. The method of claim 1, further comprising a formulation of one or more ingredients from this group consisting essentially of: green tea, curcumin, garden cress, papaya, zinc, barium, calcium and their equivalents.

11. The method of claim 1, wherein the composition further comprises an antacid selected from the group consisting essentially of: NaHCO₃, KHCO₃, CaCO₃ MgCO₃, CHNaO₃, Al(OH)₃, Mg(OH)₂, C₇H₅BiO₄, (OH)₃, Citric acid, Na₂CO₃, and combinations thereof.

12. The method of claim 1, wherein the composition regulates biological phenomena selected from the group consisting essentially of: the Na—K ATPase system, acid production in the stomach, intra-organellar acidification of intracellular organelles; urinary acidification, bone resorption; fertility; angiogenesis; cellular invasiveness; tumor cell invasiveness); metastasis; the development of drug resistance in tumor cells; cancer; osteoporosis; Alzheimer's disease, glaucoma, abnormal urinary acidification; inhibition of the entry of viruses (e.g., baculoviruses and retroviruses), inhibition of the entry of protein toxins (e.g., diphtheria toxin), into cells, inhibit fertility in an animal, for example, a human, inhibition of the invasiveness or metastasis of tumor cells, promotion of the sensitivity of cancer toward drugs by inhibiting the ability of cancer cells to develop resistance to drugs and facilitating the chemotherapeutic treatment of cancer.

13. The method of claim 1, wherein the composition is contained in an oral delivery system extending the release of the clinically significant concentrations of the composition and/or enhancing absorption into the bloodstream by releasing the composition at two or more stages of the gastro-intestinal (GI) tract by the design of the delivery system, wherein the oral delivery system includes a first clinically in-active carrier material that facilitates dissolution and absorption of the first, second, or third layer in the stomach and a second clinically in-active carrier material that facilitates dissolution and absorption of the first, second, or third layer in the small or large intestine and may include a third clinically in-active carrier material that facilitates dissolution and absorption of the first, second or third layer in the large intestine.

14. A method of treating excess stomach acid, inflammation or cancer comprising a single or a combination of active ingredients contained in an oral delivery system extending the release of the clinically significant concentrations of the active ingredients and/or enhancing absorption into the bloodstream by releasing the active ingredients at two or more stages of the gastro-intestinal (GI) tract by the design of the delivery system, wherein the oral delivery system includes a first clinically in-active carrier material that facilitates dissolution and absorption of the first, second, or third layer in the stomach and a second clinically in-active carrier material that facilitates dissolution and absorption of the first, second, or third layer in the small or large intestine and a third clinically in-active carrier material that facilitates dissolution and absorption of the first, second or third layer in the large intestine.

15. The method of claim 14, wherein the active ingredients are coated with polysebacic acid and an outer coating of polyethylene glycol to facilitate transport through the mucus lining of the GI track.

16. The method of claim 14, wherein the active ingredients include isothiocyanate or thiocyanates and the residence time of the active ingredients is extended by incorporating the active ingredients into a muco-adhesive delivery system formed by ionotropic gelation of gellan beads with gellan gum.

17. The method of claim 14, wherein the active ingredients comprise hydrophobic actives that are loaded into compounds to facilitate bioavailability in the aqueous system of the body.

18. The method of claim 14, wherein the first, second, or third clinically in-active carrier material comprise a delivery system of positively-charged gelatin microspheres for the active ingredients to substantially dissolve in the acid of the stomach for topical treatment or absorption into the bloodstream.

19. The method of claim 14, wherein the first, second or third clinically in-active carrier material contains methylmethacrylate polymer Eudragit L that dissolves substantially only in the neutral pH of the small intestine to releasing the active ingredients for topical treatment or absorption into the blood stream.

20. The method of claim 13, wherein the first, second or third clinically in-active carrier material comprises a higher concentration of the of methylmethacrylate polymer Eudragit L to further delay dissolution until this layer reaches the large intestine to release one or more actives for topical treatment and absorption into the blood stream or the first, second or third clinically in-active carrier material compose a different carrier material that takes longer to dissolve in a neutral pH environment at a lower concentration.
